(19) United States
(12) Patent Application Publication
(10) Pub. No.: US 2024/0369554 A1
(43) Pub. Date: Nov. 7, 2024

(54) METHOD OF DETECTING AND IDENTIFYING A MICROORGANISM

(71) Applicant: University of Cape Town, Cape Town (ZA)

(72) Inventors: Nicolette Rebecca HENDRICKS-LEUKES, Cape Town (ZA); Jonathan Michael BLACKBURN, Cape Town (ZA)

(73) Assignee: University of Cape Town, Cape Town (ZA)

(21) Appl. No.: 18/686,057

(22) PCT Filed: Aug. 24, 2022
(86) PCT No.: PCT/IB2022/057927

§ 371 Date: Feb. 23, 2024

(30) Foreign Application Priority Data
Aug. 24, 2021 (GB) 2112132.2

(51) Int. Cl. G01N 33/569; A61K 45/06; G01N 21/65; G01N 33/543

(52) U.S. Cl. CPC G01N 33/5695; A61K 45/06; G01N 21/658; G01N 33/5438; G01N 33/56983; G01N 2333/35

(57) ABSTRACT

A method of detecting and identifying an analyte in a sample is provided. The method comprises the steps of applying a sample to a Surface Enhanced Raman Scattering (SERS)-active surface comprising an electrode which is coated with polyelectrolyte-wrapped nanometallic particles; applying, in a step-wise manner, a first voltage and then a second voltage to the SERS-active surface; generating a SERS spectrum of the SERS-active surface; and determining whether the generated SERS spectrum is characteristic for a target analyte. The method can be used to diagnose and treat an infection. A SERS-active surface, kit and computer-implemented method for performing the above method are also provided.

### CROSS-REFERENCE TO RELATED APPLICATION

This application claims priority from United Kingdom patent application number 2112132.2 filed on 24 Aug. 2021, which is incorporated by reference herein.

### FIELD OF THE INVENTION

The invention provides a method for detecting and identifying a microorganism using electrochemical Surface-Enhanced Raman Scattering (EC-SERS).

BACKGROUND TO THE INVENTION

The need to decentralize and simplify the detection of infectious disease-causing microorganisms toward point-of-care (POC) diagnostics is becoming increasingly more important.

Among the bacterial-based pathogens, mycobacteria tuberculosis (MTB) remains the leading cause of death worldwide. WHO has endorsed an “end TB strategy” for which rapid diagnostics have been recognized as a key aspect to controlling MTB. The focus is on decentralizing the detection of MTB pathogens, ideally at the periphery level (i.e. point of care settings), while at the same time the diagnostic method must be cost effective, rapidly responsive and sensitive. Many countries still largely depend on centralized methods for MTB diagnosis, including symptom-based screening, smear microscopy, and culture media testing. These methods have a long turn-around-time and lack sensitivity. Although newer assay-based methods such as Xpert Mycobateria/MTB-RIF (Xpert), MTB/RIF Ultra, and urine lateral flow lipoarabinomannan (LF-LAM) have higher sensitivity and accuracy, they still have disadvantages (such as requiring the extraction of target MTB-markers, specialized reagents, constant electricity supply, and/or refrigerated storage conditions). Consequently, the high costs and other compounding requirements of these newer methods restricts their implementation/use in routine TB diagnosis in low resource settings. A need therefore still exists for a simple to use, sensitive and affordable diagnostic method for MTB and other microbes with true POC capabilities and compatibility with multiple biological media.

Surface Enhanced Raman Scattering (SERS) has shown potential for detecting and differentiating between bacteria-based pathogens. The inherent specificity granted by the Raman scattered photons and signal enhancement from the nanoscale metallic features enable unique spectra of molecular vibration which are highly target specific, and thus useful for “chemical fingerprinting”. Most of the SERS-based publications describing the detection of different classes of bacterial pathogens focus on either indirect (i.e. extrinsic detection using Raman reporter molecules) and/or direct detection of isolated/extracted cell constituents (from lysed bacterial cells) as target markers.

Furthermore, classical EC-SERS based spectroelectrochemical approaches with pre-formed nanometallic substrates for detecting microorganisms usually require pre-treatment with chaotropic ions in order to displace capping agents from the surface of nanoparticles. This passivates the nanometallic surface and thus reduces the SERS activity of the nanometallic feature. The pretreatment is then often followed by an incubation period of up to 16 hours in order to obtain a viable SERS signal during the follow-up electrochemical step. Additionally, reported techniques commonly employ an approach based on oxidation-reduction cycling (ORC), in which the potential is scanned (or swept) between two vertex potentials, with voltages between the vertex potentials also being scanned. This method generates SERS-active nanoclusters via roughening of the surface of the working electrode, but these are highly irreproducible and thus not conducive to creation of analytical biosensor platforms for discriminative detection in a point of care setting, where substrate-to-substrate reproducibility is an important factor.

There is therefore a need for a method of detecting microorganisms which overcomes at least some of the problems associated with existing techniques such as long turn-round times, lack of reproducibility, and the need for pretreatment procedures for displacement of capping agents.

### SUMMARY OF THE INVENTION

According to a first aspect of the invention, there is provided a method of detecting a microorganism in a sample, the method including the steps of:
applying a sample to a Surface Enhanced Raman Scattering (SERS)-active surface comprising an electrode which is coated with a film of polyelectrolyte-wrapped noble metal nanoparticles;
applying a first voltage and then a second voltage in a step-wise manner to the SERS-active surface;
generating a SERS spectrum of the SERS-active surface; and
determining whether the generated SERS spectrum or part thereof is characteristic for the microorganism.

The first voltage may be a higher voltage than the second voltage.

The first voltage may be an anodic voltage and the second voltage may be a cathodic voltage.

The method may further comprise the step of allowing the microorganism to be captured to the SERS-active surface if the microorganism is present in the sample, prior to the step of applying the first and second voltages.

The film of polyelectrolyte-coated nanometallic particles may be functionalised with capture agents which specifically recognise and capture the microorganism onto the SERS-active surface. The capture agents may be selected from the group consisting of antibodies, affibodies, enzymes, ankyrin repeat proteins, armadillo repeat proteins, nucleic acid aptamers, peptides, carbohydrate ligands, synthetic ligands and synthetic polymers.

The noble metal nanoparticles may be silver nanoparticles.

The polyelectrolyte may be poly(diallyldimethylammonium chloride) (PolyDADMAC).

The first voltage may be less than or equal to +600 mV, such as about +200 mV.

The second voltage may be less negative than or equal to −300 mV, such as about −150 mV.

The electrode may be a working electrode of a screen printed electrode (SPE).

The step of determining whether the generated SERS spectrum or part thereof is characteristic for the microorganism may be performed by comparing the generated SERS spectrum to a reference SERS spectrum of the target microorganism, or by identifying one or more vibrational mode bands in the generated SERS spectrum which are known to be characteristic for the target microorganism.

The microorganism may be a bacterium, such as Mycobacterium tuberculosis; a virus; or a parasite.

The sample may be from a human or animal, and may be a sputum sample.

The method may be performed in the absence of the SERS-active surface having been modified with a self-assembled monolayer (SAM).

According to a further aspect of the invention, there is provided a SERS-active surface including an electrode which is coated with at least a first film of polyelectrolyte-wrapped noble metal nanoparticles.

The polyelectrolyte wrapping the metal nanoparticles may be poly(diallyldimethylammonium chloride) (PolyDADMAC).

The electrode may be coated with a second polyelectrolyte film including polystyrene sulfonate (PSS).

Neither the electrode nor the nanoparticles may be coated with a self-assembled monolayer (SAM).

The nanoparticles may be silver nanoparticles.

The polyelectrolyte-wrapped noble metal nanoparticles may be functionalised with capture agents which specifically recognise a target microorganism, such as antibodies, affibodies, enzymes, ankyrin repeat proteins, armadillo repeat proteins, nucleic acid aptamers, peptides, carbohydrate ligands, synthetic ligands and synthetic polymers.

The SERS-active surface may be a modified working electrode of a screen-printed electrode.

According to a further aspect of the invention, there is provided a screen printed electrode including a working electrode, reference electrode and counter electrode, wherein the working electrode is a SERS-active surface as described above.

According to a further aspect of the invention, there is provided a computer implemented method of detecting a microorganism in a sample, the computer performing steps including:
receiving inputted subject data comprising a SERS spectrum of a sample;
comparing the data from the SERS spectrum obtained from the sample to reference data from a SERS spectrum of a target microorganism and thereby determining whether the target microorganism is present in the sample; and
displaying information regarding the presence or absence of the target microorganism in the sample.

The SERS spectrum may be obtained or may have been obtained by applying the sample to a SERS-active surface comprising an electrode which is coated with a film of polyelectrolyte-wrapped noble metal nanoparticles; applying a first voltage and then a second voltage in a step-wise manner to the SERS-active surface; and generating a SERS spectrum of the SERS-active surface.

According to a further aspect of the invention, there is provided a kit including:
at least one SERS-active surface or electrode described above; and
instructions for performing the method described above.

The kit may further comprise any one or more of the following:
a buffer solution and/or buffer-based supporting electrolyte;
a capture agent;
a reference SERS spectrum or distinguishing SERS band information of a target microorganism(s); and/or
means for collecting a sample.

According to a further aspect of the invention, there is provided a method of producing a SERS-active surface for use in detecting a microorganism in a sample by the method described above, the method including coating an electrode with at least a first film of polyelectrolyte-wrapped noble metal nanoparticles.

The polyelectrolyte wrapping the metal nanoparticles may be poly(diallyldimethylammonium chloride) (PolyDADMAC).

The nanoparticles may be silver nanoparticles.

The method may include the further step of coating the electrode with a second polyelectrolyte film comprising or consisting of polystyrene sulfonate (PSS).

Typically, the method does not include a step of coating the electrode or the nanoparticles with a self-assembled monolayer (SAM).

The method may also include the further step of functionalising the polyelectrolyte-wrapped noble metal nanoparticles with capture agents which specifically recognise a target microorganism, such as antibodies, affibodies, enzymes, ankyrin repeat proteins, armadillo repeat proteins, nucleic acid aptamers, peptides, carbohydrate ligands, synthetic ligands and synthetic polymers.

The electrode may be a modified working electrode of a screen-printed electrode.

According to a further aspect of the invention, there is provided a method of diagnosing and optionally also treating an infection of a pathogenic microorganism in a subject, the method including the steps of:
applying a biological sample to a SERS-active surface comprising an electrode which is coated with a film of polyelectrolyte-wrapped noble metal nanoparticles;
applying a first voltage and then a second voltage in a step-wise manner to the SERS-active surface;
generating a SERS spectrum of the SERS-active surface;
determining whether the generated SERS spectrum or part thereof is characteristic for the microorganism; and
if the generated SERS spectrum or part thereof is characteristic for the microorganism, making a diagnosis that the subject is infected with the microorganism;
and optionally administering an effective amount of a medicament for treating the infection to the subject.

### BRIEF DESCRIPTION OF THE FIGURES

FIG. 1: shows a carbon screen printed electrode, (a) prior to and (b) after a working electrode (WE) has been coated with a film of polyelectrolyte-wrapped silver nanoparticles. RE—Reference electrode; WE—working electrode; CE—counter electrode.

FIG. 2: shows customized multiplexing carbon screen printed electrodes, i.e., with four carbon working electrode surfaces on a single screen printed electrode (left); and with a customized tandem 4-screen printed carbon working electrode arrangement (right).

FIG. 3: shows a schematic diagram for an embodiment of the method for detecting a microorganism according to the invention (EC-SERS approach-1 (incbECSERS-1)).

FIG. 4: shows a schematic diagram for an alternative embodiment of the method for detecting a microorganism according to the invention (EC-SERS approach-2 (instECSERS-2)).

FIG. 5: shows UV-Vis spectra of silver nanoparticles (AgNP) and polyelectrolyte-wrapped AgNP (peAgNP). Inset: TEM micrograph of the peAgNP taken at high magnification. The estimated thickness of the polyelectrolyte layer (1 nm) is illustrated.

FIG. 6: shows EC-SERS-Approach-1 (incbECSERS-1), with SERS spectra of the nano-peAgSPc platform before (I a) and after exposure to the Bacillus Calmette-Guérin (wtBCG) mycobacteria (I b). Pre-incubation with the wtBCG was done in TrisHCl buffer (pH 8.5). (II c)-(II e) show potential-dependent evolution of the SERS spectra preceding and during the sequence-based positive-to-negative voltage stepping protocol. EC-SERS for incbECSES-1 was done in neat TrisHCl buffer as supporting electrolyte. (c) shows the spectrum recorded at open circuit potential. For the sequence-based potential stepping: (d) shows the spectrum recorded at +200 mV (i.e. the first voltage within the sequence and (e) illustrates the spectrum recorded at the cathodic stepping of the potential (i.e. −150 mV). The * denotes mode from nano-peAgSPc BG. The ↓ denotes disappearance of the BG-related mode band under the influence of the negative voltage. All spectra in the main graph are offset vertically for visualization. Insets exhibit the increase in band peak-intensity as a function of time for EC-SERS spectra recorded at −150 mV for up to 120 s for all major vibrational mode bands, i.e. detailed expansion of the various differentiable/represented modes (i.e., at a higher scale resolution). Excitation wavelength was 785 nm; power at the sample was 25 mW and the spectral acquisition time was 15 second.

FIG. 7: shows the voltage-dependent evolution of SERS spectra for the EC-SERS-approach-2, i.e. “instECSERS-2” (no preincubation), conducted in Tris-HCl buffer containing the wtBCG mycobacteria. SERS spectra were vertically offset for easier viewing. (c) shows the spectrum recorded at the initial polarization/pre-conditioning step (i.e. at −950 mV). For the sequence-based potential stepping: (d) shows the EC-SERS spectrum recorded during application of the positive voltage (at +200 mV, i.e. the first voltage within the sequence); (e) illustrates the spectrum recorded at the cathodic stepping of the potential (i.e. −150 mV). All displayed spectra have been normalized for both laser power and acquisition time. All spectra were recorded in pH 8.5-8.8 Tris buffer supporting electrolyte which contained the target bacteria. During voltage stepping the applied potential was maintained throughout acquisition. The excitation wavelength was 785 nm, power at the sample was 25 mW and the spectral acquisition time was 15 s.

FIG. 8: shows SERS signal for mycobacterial wtBCG obtained through incbECSERS-1 (pre-incubation followed by EC-SERS), for 3 separate trials on 3 separate nano-peAgSPc platforms ((a)-(c)). Spectra were recorded in air after rinsing/drying of electrodes. Inset: Comparative SERS spectral pattern for wtBCG obtained from the instECSERS-2 (EC-SERS-approach-2). Each exhibited spectrum is an average of multiple individual spectra taken across the SERS substrate. For each separate trial represented, the SD of the averaged spectra at each spectral point (±1σ) is given by the shaded region for each spectrum. Data was collected using a 785 nm laser line with a laser power of 25 mW and an acquisition time of 15 s.

FIG. 9: shows the effects of variation of culture conditions on the reproducibility of the obtained SERS spectral signature for mycobacteria bovis BCG (wtBCG) and scanning electron microscopy (SEM) characterization of the nano-peAgSPc with the immobilized mycobacteria. (a): representative SERS spectrum of wtBCG on the nano-peAgSPc, in which the preceding mycobacterial culturing was done in polysorbate-free 7H9 media (referred to herein as 7H9-media-A). (b): representative SERS spectrum of wtBCG on the nano-peAgSPc, in which the preceding mycobacterial culturing was done in 7H9 media containing polysorbate-80 (referred to herein as 7H9-media-B). For each SERS spectrum, the SD is represented by the shaded area. SEM images illustrating the nano-peAgSPc with the mycobacterial cells in which the wtBCG was derived from 7H9-media-B is shown in graphs I-iii for 5.0 (i); 40 (ii); and 100 kx (iii) magnifications, respectively. iv-v show the SEM images of the immobilized mycobacterial bacilli on the nano-peAgSPc, in which the wtBCG was derived from 7H9-media-A.

FIG. 10: shows characteristic SERS spectral pattern of wtBCG obtained on the nano-peAgSPc substrate showing all major bands at high scale resolution (a); and a comparative SERS spectrum of the control, i.e. SERS spectrum obtained in the absence of any probe bacteria (b). Both SERS spectra were recorded after completion of the sequence-based electrochemical voltage stepping protocol, in air after rinsing and drying the substrates. Each exhibited spectrum is a mean spectrum, averaged from multiple (at least 12-15) individual spectra. All spectra are normalized.

FIG. 11: shows a SERS spectral response for TB-H37Rv as a function of incubation and EC-SERS: SERS spectra (I) of the nano-peAgSPc platform before (a) and after exposure to the TB-H37Rv strain of mycobacteria (b). SERS spectra recorded in mycobacteria-free supporting electrolyte at open circuit potential (c) and under the influence of the cathodic/negative voltage, i.e. −150 mV (d). The supporting electrolyte used during EC-SERS was Tris-HCl (pH 8.5). Pre-incubation of the nano-peAgSPc platform with the TB-H37Rv mycobacteria was done in TrisHCl buffer. (a)-(b) show SEM images of TB-H37Rv on the surface of the silver nanometallic platform, taken at different magnifications.

FIG. 12: shows signal reproducibility studies for H37Rv on the nano-peAgSPc platforms with the EC-SERS technique: (a): SERS signature for TB-H37Rv obtained through incbECSERS-1 for 3 separate trials on 3 separate nano-peAgSPc platforms (the SD of the mean spectrum in each case illustrated as the shaded area). (b): Spot-to-spot SERS spectral comparison of TB-H37Rv on the surface of the nano-peAgSPc. Laser excitation was 785 nm. Power at the sample was 25 mW and acquisition time was 15 s.

FIG. 13: shows the characteristic SERS spectral signature for TB-HN878 (a); and for TB-CDC1551 mycobacteria (b), obtained through incbECSERS-1, for 2 separate trials, each with 2 separate nano-peAgSPc platforms. For each SERS spectrum, the standard deviation (SD) is represented by the shaded area. Data was collected using a 785 nm laser line with a laser power of 25 mW and an acquisition time of 15 s.

FIG. 14: shows a comparison of the SERS vibrational signatures for tested strains of mycobacterium tuberculosis, i.e. H37Rv, HN878 and CDC1551, shown at a higher scale resolution. Power at the sample was 25 mW; acquisition time was 15 seconds. Each vibrational signature is a mean spectrum averaged from multiple individual spectra.

FIG. 15: shows SERS signatures of all mycobacteria used in this investigation, i.e. wtBCG, TB-H37Rv, TB-HN787 and TB-CDC1551, and their spectral pattern comparison with the SERS vibrational signatures of gram-positive Staphylococcus aureus (S.A.) and gram-negative E. coli (K12) bacteria.

FIG. 16: shows a response of a functional biosensor chip, with antibody (Ab-nano-pe₂AgSPc) for EC-SERS based detection of wtBCG, released from HIV_{neg} liquefied sputum (Plot a), HIVₚₒₛ liquefied sputum (Plot c), from urine (Plot b); and directly from completely un-processed HIV_{neg} sputum (Plot d). For these studies the neat clinical biological medium/matrix for each sample was spiked with the wtBCG (obtained via culture). For the sputum liquefaction process, NaOH-based alkaline liquefaction procedure was used, including mucolytic neutralization and centrifugal collection of released tubercle bacilli. For comparison, Plot e illustrates the SERS signal obtained directly from culture grown wtBCG (i.e. not spiked into any biological matrix).

### DETAILED DESCRIPTION OF THE INVENTION

A method of detecting and identifying an analyte in a sample is described herein. The method comprises the steps of applying a sample to a Surface Enhanced Raman Scattering (SERS)-active surface including an electrode which is coated with polyelectrolyte-wrapped nanometallic particles; applying, in a step-wise manner, a first voltage and then a second voltage to the SERS-active surface; generating a SERS spectrum of the SERS-active surface; and determining whether the generated SERS spectrum is characteristic for a target analyte. A SERS-active surface, kit and computer-implemented method for performing the above method are also described.

A SERS spectrum is essentially an amplified Raman spectrum of the target molecule. The generated spectrum consists of a series of peaks or vibrational mode bands, which are fingerprints of the target molecule and provide a unique vibrational signature of the target species. The signal enhancement/amplification is mainly due to the electromagnetic interaction of the incident light (from a monochromatic light source) with a SERS-active metal, which produces large amplifications of the laser field through excitations, which are generally known as plasmon resonances. The SERS-active metal is in the form of metallic nanoparticles, ideally of a noble metal. As SERS is a surface spectroscopy technique, the target molecules should be in very close proximity to the nanometallic surface (preferably within 10 nm), and should thus ideally be adsorbed on the nanometallic surface. During EC-SERS, the applied voltage can be used to increase the fermi level of the metal, resulting in further amplification of signal.

The method described herein is a new electrochemical SERS (EC-SERS) technique which is based on a combination of vibrational spectroscopy and electrochemistry (i.e., spectroelectrochemistry). The nature of EC-SERS is such that surface enhanced Raman scattering spectra are recorded at the interface of an electrochemical (EC) double layer (DL). The interface of the EC DL refers to the solid-liquid junction between the surface of a nanometallic feature (of atomic scale roughness) and the liquid from a supporting electrolyte.

The nanoparticles used herein can be noble metal-based, such as silver or gold nanoparticles, and further may be isotropic (pseudo-spherical) or anisotropic nanoparticles.

The polyelectrolyte (PE) that is wrapped around the nanoparticles can be a cationic polyelectrolyte, such as poly(diallyldimethylammonium chloride) (PolyDADMAC) or poly(allylamine hydrochloride) (PAH).

The film of polyelectrolyte-coated nanometallic particles can be functionalised (decorated) with capture agents which specifically recognise and capture the analyte onto the SERS-active surface. The capture agents can be antibodies, affibodies, enzymes, ankyrin repeat proteins, armadillo repeat proteins, nucleic acid aptamers, carbohydrate ligands, synthetic ligands or synthetic polymers which specifically recognise and bind the target microorganism, so as to confer selectivity to target organisms. In one embodiment, the capture agents are antibodies, and more particularly are monoclonal antibodies.

Prior to the step of applying the first and second voltages, the analyte can be allowed to be captured to the SERS-active surface if it is present in the sample, either by means of a capture agent described above or by adsorption. An incubation step can be performed to allow this to occur. The incubation step can be for a period of an hour or less, such as less than 40 minutes, less than 20 minutes, less than 10 minutes, from 5 to 10 minutes, or for about 5 minutes.

Optional washing and drying steps can be performed to remove sample that has not been adsorbed or captured to the SERS-active surface before the anodic and cathodic voltages are applied.

The SERS-active surface includes an electrode which is at least partially coated with polyelectrolyte-wrapped noble-metal nanoparticles. The SERS-active surface does not have to be further modified with a self assembled monolayer (SAM) in order for the analyte to be detected by the method described herein. Thus, the PE-wrapped nanoparticles can be coated directly onto the electrode, without an intervening SAM between the electrode and the PE-wrapped nanoparticles. The electrode can be a working electrode of a three-electrode system, such as a screen printed electrode (SPE).

The first and second voltages can be applied to the electrode(s) with the aid of a potentiostat. The first voltage that is applied to the SERS-active surface can be a higher voltage than the second voltage, on a scale running from high positive voltages at the high end of the scale to high negative voltages at the low end of the scale. The first voltage can be an anodic voltage and the second voltage can be a cathodic voltage.

The anodic voltage that is applied to the SERS-active surface is typically less than or equal to about +600 mV, such as less than about +500 mV, less than about +400 mV, less than about +300 mV, about +200 mV or about +100 mV. For example, the anodic voltage that is applied can be in the range of from +150 mV to +300 mV, and more particularly in the range of from +150 mV to +200 mV.

The cathodic voltage that is applied to the SERS-active surface is typically less negative than or equal to about −350 mV, such as less negative than about −300 mV, less negative than about −200 mV, about −150 mV or about −100 mV. For example, the cathodic voltage that is applied can be in the range of from −50 mV to −200 mV, and more particularly in the range of from −100 mV to −200 mV. In one embodiment, the cathodic voltage is −150 mV.

The step of determining whether the generated SERS spectrum is characteristic for a target analyte can be performed by comparing the generated SERS spectrum with a reference SERS spectrum of the target analyte, or by identifying one or more (e.g. two or three) vibrational mode bands (peaks) in the SERS spectrum which are known to be characteristic (i.e. unique) for the target analyte.

The analyte can be a microorganism. The microorganism can be any pathogenic microorganism, such as a bacterium, virus or parasite. Examples of these include gram negative bacteria, gram positive bacteria, tuberculosis-derived mycobacteria (e.g. Mycobacterium tuberculosis, M. bovis, M. avium, etc.), Streptococchus aureus, Escheriscia coli, Salmonella entericha, Salmonella typhi, Vibrio cholerae, HIV, Covid-19, helminths such as schistosomes, and so forth.

The sample can be a biological sample from a human or animal, and for example can be a sputum sample, blood sample (e.g. whole blood, serum or plasma), saliva sample, urine sample or stool sample. Alternatively, the sample can be from a substance suspected of being contaminated with a microorganism, such as food or water. The sample can be suspended in an electrolyte solution prior to being applied to the SERS-active surface.

The method can be performed without pretreating the SERS-active surface with chaotropic ions (e.g. Cl) before the sample is applied.

If a target microorganism is identified in a sample, the subject from whom the sample was obtained can be treated for an infection by that microorganism, e.g. by administering an anti-viral composition, anti-bacterial composition or anti-parasitic composition to the subject. For instance, if Mycobaterium tuberculosis is identified in the sample, the subject can be administered an effective amount of a medicament which is suitable for treating tuberculosis infection or Tuberculosis Disease. Similarly, if HIV is identified, the subject can be administered with an antiretroviral or other HIV medication.

A SERS-active surface or (bio) sensor is also described herein. As used herein, a “SERS-active surface” refers to an electrically conductive support or electrode which is at least partially modified with a SERS substrate (SERS-active nanoparticles), and is sometimes referred to in the art as a SERS platform, SERS biosensor or SERS nanochip. SERS or EC-SERS is performed on this SERS-active surface. An electrode is a conductor that is used to make contact with a nonmetallic part of a circuit. The electrically conductive support can comprise a three electrode system having a working electrode, counter (or auxiliary) electrode and reference electrode. The working electrode is the electrode on which the reaction of interest will occur. Common working electrodes can consist of materials ranging from inert metals such as gold, silver or platinum, to inert carbon (such as glassy carbon, boron doped diamond or pyrolytic carbon).

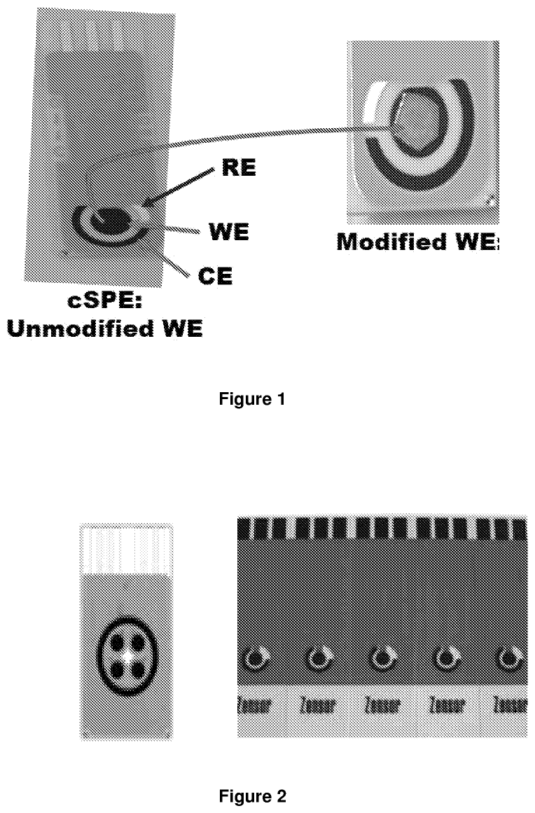

One example of a three electrode system is a screen printed electrode (SPE). Disposable carbon screen printed electrodes (cSPE) were selected as the solid support for one embodiment of the SERS-active surface because they are readily available and relatively inexpensive. Each of these screen printed electrodes has at least one of each of an integrated working electrode (WE), a counter electrode (CE) and a silver reference electrode (pseudo reference) (FIG. 1). The SERS-active surface can also comprise two or more (i.e. multiple) working electrode surfaces to allow for a multiplexing assay to be performed (FIG. 2). This will allow samples from two or more subjects to be tested at the same time, or allow a sample to be tested for two or more target microorganisms (or species thereof) at the same time (e.g. by conjugating different capture agents to each working electrode).

At least the working electrode is modified or coated with the SERS-active substrate, in the present case comprising an optically transparent film of polyelectrolyte-wrapped nanometallic particles as described above. The electrode can also be coated with a second polyelectrolyte film, such as polystyrene sulfonate (PSS). Capture agents which specifically recognise and capture the microorganism can be conjugated onto the SERS-active surface.

A computer implemented method of detecting a microorganism or other analyte in a sample is also described, wherein the computer performing steps comprise:
receiving inputted subject data comprising a Surface Enhanced Raman Scattering (SERS) spectrum of a sample;
comparing the data from the SERS spectrum of the sample with reference data from a SERS spectrum of a target microorganism or analyte and thereby determining whether the target microorganism or analyte is present in the sample; and
displaying information regarding the presence or absence of the target microorganism or analyte in the sample.

A kit is further provided, which comprises at least one SERS-active surface described above and one or more of the following:
means for collecting a sample;
instructions for performing the method described above;
one or more buffer solutions and/or buffer-based supporting electrolytes;
one or more capture agents;
one or more reference SERS spectra or distinguishing SERS band information of target microorganism(s) or analytes; and/or
means for collecting a sample.

When preformed nanoparticles are used for SERS detection of microorganisms, especially in the case of bacterial-based microbial pathogens, the target pathogen needs to be adsorbed onto the nanoparticle surface, since the nature of the SERS phenomenon is such that the decay of the SERS-based signal enhancement is distance-dependent (thus requiring intimate interaction between the surface of the nanoparticles and the target microorganism).

However, highly pathogenic microorganisms such as TB mycobacteria generally have a high degree of waxiness within their cell wall or envelope. Detection platforms which use charge-based interaction as the predominant or only approach of interaction are therefore not suitable for detecting these microorganisms.

During the natural process of biofilm formation or adsorption of microorganisms onto a substratum, there is usually a layer on the substratum onto which the microorganism adsorbs (referred to as a conditioning layer). The SERS biosensor described herein was designed to mimic this process, and the polyelectrolyte film was selected and developed so as to function as a pseudo conditioning film to enhance the affinity for the target microorganism. Moreover, microbial bio-adhesion is also highly dependent on hydrophobic-hydrophilic properties of the interacting surfaces. Polyelectrolytes can foster both charge-and hydrophobic-hydrophilic based interactions with microbial targets, and were therefore investigated for their suitability for tethering captured microorganisms to substrates.

In one embodiment of the invention, the polyelectrolyte (PE) is poly(diallyldimethylammonium chloride) (PolyDADMAC). PolyDADMAC is a polyamine-type PE and has its cationic charges along the backbone of the polymer, and not as pendent side groups as seen in many other polymer types. The dimethylamine of PolyDADMAC is easily replaced by other amines, such as those in adenine (which is found in bacteria). PolyDADMAC also results in hydrophobic colloidal coagulation and bridging-adsorption, as well as having rheological properties, all of which were found to be beneficial for adsorption of microorganisms during substrate adsorption and/or biofilm formation.

The nanoparticles can be mixed into the polyelectrolyte and wrapping can be allowed to occur.

PE wrapping of the synthesized nanoparticles generally proceeds for 20-30 min in the presence of a chaotropic anion (Cl⁻), e.g. by using NaCl. After wrapping, excess polyelectrolyte can be removed by centrifugation. In one embodiment, at least 2-3 rounds of centrifugal washing at 7500 rpm is performed to ensure removal of excess polyelectrolyte. The thickness of the film of polyelectrolyte on the nanoparticles is typically less than or equal to 1 nm.

Following the final centrifugal wash, the supernatant is removed and an aqueous concentrated nanoparticle suspension is prepared. The carbon working electrode of a screen printed electrode, which can be commercially obtained, is then modified with an aliquot of this suspension.

Unlike other methods involving PE-wrapped nanoparticles, the SERS-active surface described herein is not modified with a self-assembled monolayer (SAM) prior to being coated onto the surface of the working electrode. Without this intervening SAM layer, the target microorganism was found to more intimately interact with the nanoparticles.

Nanoparticles are generally prepared in the presence of a stabilizing or capping agent (such as citrate), which leads to a ligand on the surface of the nanoparticles. This ligand needs to be displaced from the surface of the nanoparticles to enable interaction with the target species. Ligands can be displaced in several different ways, such as by reductive desorption or pre-treatment with chaotropic ions. Inorganic salts such as NaCl are usually used for this purpose, but the chloride ions have such a high binding affinity for the nanoparticles that they are difficult to displace. Also, pre-treatment for displacement or removal of the ligand from the nanoparticle surface in the context of a SERS nanometallic film generally results in a degree of passivation of the nano-substrate, which leads to lowering of the SERS signal intensity. Moreover, lengthy incubation periods with the biological target are usually required on SERS substrates that have been Cl⁻ pre-treated in order to obtain a viable signal.

However, because the polyelectrolyte that is on the surface of the nanoparticles in the present invention favorably interacts with the target microorganism, it also functions as a promoter (thus not damping the SERS signal). The nanoparticles described herein can therefore be pre-synthesized without a stabilizing/capping agent.

The activation energy of desorption of bacteria on substrata is generally low, and to effect irreversible adsorption of target bacteria, numerous studies focusing on achieving SERS-based detection of bacteria have therefore adopted a layer-by-layer PE-coating approach, by coating both the bacteria and the nanoparticles with multiple layers of charged PE in order to try and achieve the intimate contact required for SERS. However, this approach results in the bacteria-related modes in SERS spectra being obscured by interfering PE-based vibrational mode bands. The method described herein does not require the use of this multilayer PE approach to both biological target and nanoparticles. The applicant has found that bacterial adhesion to the SERS nanometallic platform can be sufficiently enhanced/consolidated by performing EC-SERS as described herein. The co-existing static EC-field generated in an EC-SERS system strengthens the metal-adsorbate bonding, thereby enhancing the associated specific adsorption of target species. The results clearly show that for bacterial targets the method described herein can dramatically shorten pre-incubation time (from more than 16 hours in known methods to less than half an hour), and can even preclude the need for a pre-incubation step altogether.

As mentioned above, in one embodiment the PE-coated nanoparticles are functionalised with biological recognition elements (or capture agents) which specifically recognise and bind the target microorganism, so as to confer selectivity to target organisms. In one embodiment, the capture agents are antibodies for the target microorganism. One method for conjugating the capture agents to the SERS platform is via conjugation onto the pre-formed nanometallic film using a polyelectrolyte such as polystyrene sulfonate (PSS) as linker. For this purpose, only the top layer of the nanometallic film is exposed to PSS for derivatizing the surface with the biological capture agent. When the capture agents are antibodies, the use of PSS to perform the conjugation allows/results in the antibodies being conjugated to the film in a specific manner. Binding of the antibody to the precursor film through PSS as linker, at pH 7.4, occurs largely through hydrophobic interaction with the aryl portion of the PE, and to a lesser extent via charge-based interaction with the positively charged segment of the antibody. Moreover, binding through PSS as linker ensures that the bio-specific activity of the immobilized antibody for its target antigen is maintained.

The biological sample can be suspended in an electrolyte solution prior to being applied to the SERS substrate, to:
further refine/improve the transfer efficiency of the target microorganism from aqueous suspension to the SERS surface;
speed up the encounter rate between the target and antibody molecular recognition element (where appropriate);
assist in blocking of non-specific binding from possible contaminating species, cell debris and any other potential fouling constituents from the clinical sample (where appropriate); and/or
help pull down non-sedimentable fragments of target microorganisms onto the SERS surface for detection (these may also be effective components for discriminative detection of the targeted infectious diseases).

In one embodiment described herein, the sample is a sputum sample from a patient who is suspected of being infected with a pathogenic microorganism. An initial alkaline liquefaction can be performed on the sputum to release the mycobacteria or other microorganisms. As conventional mucolytic/liquefaction neutralizing buffer (i.e. phosphate buffer saline (pH 6.8)) is a SERS-suppressant, it may potentially cause downstream damping of the SERS signal. Additional centrifugal washing of the microbial pellet can therefore be performed. Alternatively, a buffer such as 200 mMolar borate buffer (pH 6.8), can instead be used as a neutralizing buffer of the liquified sputum. This neutralizing buffer is also effective in lowering the specific gravity of the liquefied sputum solution in order for sufficient sedimentation during centrifugal collection of the released mycobacteria. The microorganism may also be detected directly from un-liquified sputum, either as is, or diluted with an appropriate capture buffer.

The method described herein can be performed with intact microorganisms, and does not require any deliberate dislodging and/or more aggressive disruption of any part of the complex cell envelope of the microorganism for extraction or isolation of target species. This markedly simplifies the detection of the microorganisms toward POC applications, such as in periphery-level clinics and other low resource-settings.

In an embodiment, prior to the sample or an isolated part thereof being placed on the SERS biosensor, the sample or part thereof can be suspended in a capture buffer (e.g. TrisHCl 200 mM-1000 mM).

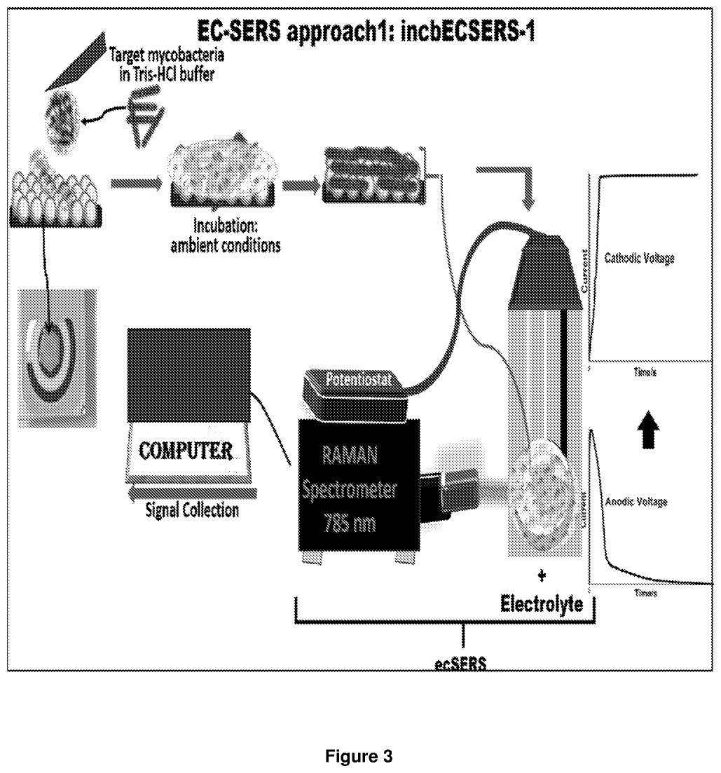

In one embodiment, the sample is incubated on the biosensor before performing EC-SERS. The incubation period is typically less than an hour, such as from about 5 min to about 40 minutes. After incubation, the biosensor is rinsed and dried and fresh supporting electrolyte (e.g. TrisHCl buffer) is applied to the integrated working, counter and reference electrodes of the biosensor. An EC-SERS voltage stepping protocol is then performed (i.e. in neat electrolyte). This embodiment is referred to herein as “EC-SERS-approach1” (FIG. 3).

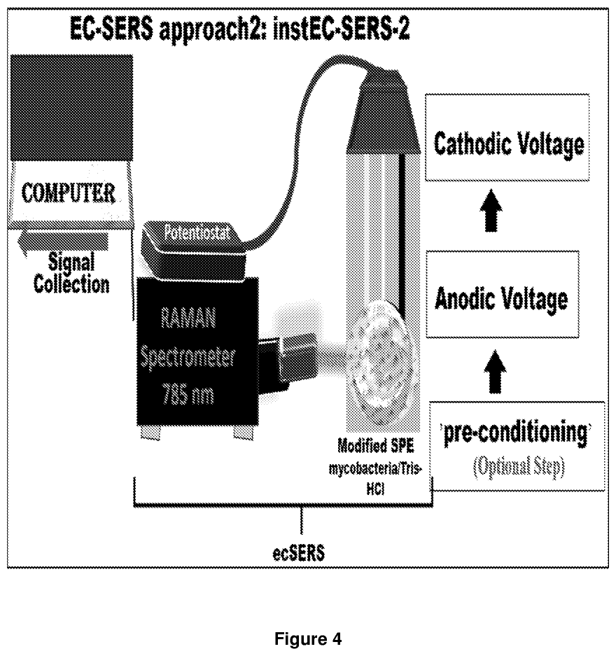

In an alternative embodiment, EC-SERS is performed without pre-incubation of the sample on the biosensor. This embodiment is referred to herein as “EC-SERS-approach2” (FIG. 4). The integrated working, counter and reference electrodes are covered by the sample/electrolyte before EC-SERS is performed (i.e. in the presence of the sample and/or microorganism). Optionally, the nanometallic film can be polarized or pre-conditioned at a negative voltage (e.g. of about −950 mV) before the sequence-based voltage stepping is commenced.

In the EC-SERS voltage stepping protocol, a first voltage is applied to the biosensor, and after steady state is attained, the voltage is stepped to a second voltage. This is performed in a step-wise manner, using potentiostatic voltammetry, and not potentiodynamic or sweeping voltammetry. In other words, the voltage is not swept across a voltage range, but rather each voltage is applied consecutively for a set time period. The optimum applied first/second voltage will depend on the type of capture agent used (if any), the species/type of targeted microorganism and the type and size of the nanoparticles. For example, in the case of TB mycobacteria, the optimum first voltage was determined to be an anodic voltage of +200 mV and the optimum second voltage was determined to be a cathodic voltage of −150 mV. Subject to not being bound by any specific mechanism of action and/or theory, it is proposed that the sequence-based positive-to-negative electrochemical voltage stepping reinforces specific and irreversible adsorption of a captured microorganism to the PE-coated nanometallic surface.

The applicant has shown that microorganisms (and even different species) have SERS vibrational signatures that are unique to that specific type/species of microorganism, and can therefore be used for both species-and strain-dependant differentiation. The appearance of characteristic bands can be seen from generated SERS spectra immediately following stepping of the applied voltage, although results are most reproducible when steady state is reached (e.g. after about 60 s and more particularly after about 100-150 s). Distinctive and vivid SERS spectra can still be obtained from biosensors months after the voltage stepping had been applied.

The SERS spectrum generated from the sample can be compared to a reference spectrum or distinguishing band(s) for the target microorganism to determine whether the target microorganism is present in the sample. Alternatively, a reference library of the SERS vibrational signatures of different microorganisms (or species thereof), or of the distinguishing bands for each microorganism or species thereof, can be established. Where there is a possibility of different microorganisms being present in the sample, the SERS spectrum generated from the sample can be compared to the reference spectra in the library (or the reference bands) to detect whether a particular microorganism is present in the sample.

Stepping the voltage to the positive direction on a preformed silver-nanoparticle based nanometallic feature would not be the first choice for a person skilled in the field of EC-SERS/spectroelectrochemistry. This is due to the potential of overoxidation of the nanoparticles and the associated fouling of the nanofilm and concomitant complete loss of SERS signal. Additionally, in EC-SERS, anodic to cathodic sequence stepping is predominantly used within electrochemical roughening/pre-activation, which involves a sweeping/scanning electrochemical approach between two vertex potentials, such as cyclic-and/or linear sweep voltammetry, usually in the process of activation of bulk silver or gold WE surfaces, aimed at generating SERS-active nanoclusters (plasmonic nanostructures) on the surface of the WE. Furthermore, with the exclusion of the recently developed electrochemical surface oxidation enhanced Raman scattering (EC-SOERS), by-and-large almost all of the EC-SERS reports involving preformed silver-based nanometallic features are based on cathodic/negative stepping of the voltage. Finally, for voltammetry based electrochemical experiments, it would usually not be preferable for someone of skill in the art to not pre-purge the supporting electrolyte due to the potential negative effects of radical species formed from the oxidation and/or reduction of ambient oxygen.

Compared to smear microscopy, which requires upwards of at least 6.5×10³ cfu/ml to be present in a sample in order to make a positive diagnosis, the method described herein is highly sensitive and inherently able to detect order of magnitude lower number of bacilli within the field of view (e.g. less than about 100 tubercle bacilli, from about 10-100 bacilli, or even less than 10 bacilli within the field of view). This is significant because in many cases, particularly with HIV co-infection and in the case of children, clinical specimens exhibit paucibacillary disease and a patient-derived specimen has order of magnitude fewer number of bacilli. These specimens therefore usually yield smear negative results and are only shown as positive via follow-up culture and/or GeneXpert Polymerase Chain Reaction (PCR) test. Moreover, clinically derived TB mycobacteria reportedly have an inherently low buoyant density, which means that it would be difficult to sediment the mycobacteria released from a clinical sample. If the sample is paucibacillary to begin with, then there are very few bacilli in the sample and great effort needs to be made in order to effectively transfer the few bacilli that can be isolated to the surface of the biosensor chip for capture and detection. In the method described herein, the surface features of the SERS substrate confer high sensitivity and reactivity for fast target capture. The concentration/ionic strength of the capture buffer has also been carefully optimized to further effect fast transfer efficiency of target to the biosensor chip.

To the best of the applicant's knowledge, this study is the first to describe EC-SERS-based detection of microorganisms using the SERS substrate described herein and/or a non-classical EC-SERS approach that includes a sequence-based positive to negative voltage stepping. The suitability of the PE-based pseudo-conditioning layer in conjunction with a sequence-based voltage stepping protocol for the specific adsorption and associated discrimination of the mycobacterial species on the silver nanometallic platform is illustrated in the examples below for 3 different strains of hypervirulent MTB. The EC-SERS platform was able to directly detect and identify TB-derived mycobacteria, without requiring any deliberate dislodging/disruption and/or extraction of any cell components. Since no additional Raman-active labels were required, thereby enabling direct detection of the target, the EC-SERS technique can be considered label-free and intrinsic. Considering the simplicity of the technique, and the small volume of sample required (i.e. only approximate 80-150 μL), this EC-SERS platform demonstrates the potential for realizing POC detection and is thus a viable approach for simplifying diagnosis. Furthermore, species-and strain level discrimination of microorganism is possible.

The method will now be described in more detail by the following non-limiting examples. Although these examples primarily relate to bacteria and their properties, the invention is not intended to be limited to the detection and identification of bacteria, and can also be used to detect other microorganisms such as viruses and parasites.

### EXAMPLES

### Methods

### Reagents

High purity silver nitrate (AgNO₃, 99.999%) was purchased from SA Precious Metals™. Poly(diallyldimethylammonium chloride) (PolyDADMAC), low molecular weight cut-off); Tris(hydroxymethyl)aminomethane (ACS reagent); hydrochloric acid (HCl), nitric acid, sodium hydroxide (NaOH) and α-D-Glucose were all obtained from Sigma Aldrich™. Ammonium hydroxide (NH₄OH), 25%, was obtained from Merck Millipore™. PSS (low molecular weight cutoff) was obtained from Sigma Aldrich™ and Polysciences Inc™. Ultrapure (Milli-Q™) water with a resistivity of 18 MΩ, produced with a Millipore Synergy™ UV water purification system, was used for the preparation of all solutions. All the glassware was washed with Alconox™, and for preparation of the AgNP, were all cleaned with aqua regia and extensively rinsed with Milli-Q™ water before use. All solution purging was done using high purity nitrogen gas, obtained from Air Products SA™. A 1 Molar stock solution of Tris-HCl buffer (T-buffer), pH 8.5-8.7, was prepared and used for the preparation of the working Tris-HCl buffer solution.

### Synthesis and Polyelectrolyte Wrapping of Silver Nanoparticles

Preparation of silver nanoparticles (AgNP) was done via a one-pot synthesis method, involving the reduction of [Ag(NH₃)₂]⁺ complex cation, which is otherwise known as the modified Tollens method and involves the reduction of silver ions (Ag⁺) in the presence of ammonia, using NaOH as activator.²⁻⁴ An aldehyde, glucose, was used as the reducing agent, because it does not adsorb onto the silver surface and is easily washed away from the nanoparticle surface during post-processing, thus minimizing background interference and/or preventing potential chemical interactions. The preferred pH for the synthesis of the AgNPs was 11.5, as this pH ensured minimization of size variability between NP colloids. An inert atmosphere during the synthesis process was ensured by purging the reaction medium with high purity nitrogen gas. The silver nanoparticle formation proceeded under continuous sonication and synthesis was complete after 33-35 minutes. The temperature of the water in the bath sonicator was preferably maintained at 37-38° C. throughout the synthesis process. Excess reagent and by-products were then removed from the as-synthesized AgNP through centrifugation, which was done with 1.0 mL aliquots, at 4 500 rpm for 10 minutes. The collected pellets were re-dispersed in 1 mL Milli-Q water, followed by 1-2 repeat rounds of centrifugal washing. For the polyelectrolyte (PE) wrapping of the AgNP, multiple washed pellet concentrates were combined, followed by direct polyDADMAC (10 mg mL⁻¹) interaction in the presence of NaCl.

The PE wrapping reaction time was maintained at or below ˜30 min. After completion of this reaction, excess reagents were removed through centrifugation at 7500 rpm. The stabilization of the colloids, by the additional presence of the PE on the surface of the AgNP, enabled centrifugation at much higher rpm's than with un-modified, as-synthesized nanoparticles. Characterization of the silver nanoparticles, before and after PE-modification, was done by UV-Vis spectrophotometric analysis, based on the utility of this conventional method to ascertain colloid stability, nanoparticle surface property changes and aggregation behavior. Additionally, transmission electron microscopic (TEM) analysis of the AgNP and PE-wrapped nanoparticles was done, to determine the size and polydispersity of the nanoparticles.

### Preparation of Silver Nanoparticle Films on Screen Printed Electrodes The peAgNP pellet obtained from the above procedure was dispersed in 1 mL Milli-Q water, followed by at least three centrifugal washes to remove all excess reagents, while sufficiently thinning the PE surface coating, thus ensuring an approximate thickness of +/−10 Å for the PE layer around the AgNP surface. This ensured laser/optical transparency for use in the EC-SERS and SERS experiments without suppressing the SERS response and/or adversely affecting any bacteria-related vibrational signals. Following the final centrifugal wash, the supernatant was removed and an aqueous concentrated nanoparticle suspension was prepared by adding Milli-Q water. Two 10 μL aliquots of the concentrated nanoparticles were drop coated onto the carbon working electrode of commercially obtained screen printed electrodes (DRP-110, Dropsens™), with intermittent drying of each layer. Thus, fairly uniform nanometallic films were formed through this simple unprogrammed assembly method. Surface morphological characterization of the pre-formed nanometallic films was done via Scanning electron microscopy (SEM) analysis.

### Instrumentation

The Raman spectrometer used in this study was a DeltaNu™ benchtop dispersive Raman spectrometer (Advantage 785), equipped with an air-cooled CCD; 785 diode laser; and right angle input optics. All Raman and SERS spectra were collected through back scattering with the same optics. All electrochemical measurements were performed using a pocket-sized, usb-powered micro-potentiostat (μStat 200, Dropsens S.L., Oviedo Spain). The commercially obtained carbon screen printed electrodes (cSPE) used for preparation of the SERS-active nano-peAgSPc substrates, featured an integrated circular working electrode (4 mm diameter), a carbon counter electrode, and a silver pseudo reference electrode (silver psRE). All the potentials reported in this work are relative to the silver psRE. During experiments, the working electrode (WE) was connected to the potentiostat with a uStat cable connector (Dropsens™).

All UV-visible spectrophotometric analysis was done with a Varian™ Cary 50 scan UV visible spectrometer. Scanning electron microscope (SEM) images were collected with a FEI Nova™ SWM 230 equipped with a field emission gun (FEG). Transmission Electron Microscopic (TEM) analysis was done with a FEI™ Tecnai 20 transmission electron microscope (FEI, Einhoven, Netherlands) operating at 200 kV and fitted with a Gatam™ Tridien energy filter and Gatam™ camera (Gatam, UK), while samples were analyzed with a nitrogen-cooled double-tilt specimen holder.

### Bacteria Handling and Sample Preparation

All bacteria cultures for this study (including selected mycobacterial species, gram negative bacteria and gram positive bacteria) were grown to mid-log phase at 37° C. with constant shaking, until reaching an optical density (OD₆₀₀) of ≈0.8-1.0. For the mycobacterial species, including wild type mycobacterium bovis BCG (wtBCG), inoculation was done from glycerol stocks and cultures were grown in OADC supplemented 7H9 Difco™ media (Becton Dickinson, BD). For the signal reroducibility investigation experiments involving variation in culture conditions, growth of the wtBCG through liquid media culturing was done both in the presence and in the absence of polysorbate/Tw80. The gram negative (including Escherichia coli, K-12-strain (E. coli)) and the gram positive (including Staphylococcus epidermis (SE) and Stephylococcus aureus (SA)) bacteria cultures were grown in Luria Bertani (LB) broth. To harvest the various bacteria, approximately 1.8 mL from each culture was centrifuged at 4,600 rpm, followed by removal of the supernatant. The pellet was then re-suspended in sterile water and subjected to another round of centrifugation. This wash procedure was repeated at least three times to ensure removal of all growth media constituents. The MTB mycobacterial strains tested included H37Rv; CDC 151; and HN 878, all of which were obtained as whole cell culture pellets from BEI resources. Each MTB strain was pre-inactivated through gamma irradiation, and washed with PBS, and thus obtained by us as a highly concentrated pellet. After receipt of the stock pellets, aliquots of approximately 100 μL were removed and stored in separate cryovials, as to prevent continuous freeze-thaw cycles of stocks. Notably, considering that the MTB strains were already centrifugally washed, they could be used as is. However, to maintain consistency with lab-cultured bacteria, as well as to de-clump aggregates where required, resuspension of aliquots was initially done by brief inverted-mixing in borate buffer or 7H9 media, to the same OD₆₀₀ as for the lab-cultured other (myco) bacteria, followed by centrifugal washing. The supporting electrolyte/working T-buffer solution for the EC-SERS experiments was T-buffer, which was prepared from the 1 Molar T-buffer stock solution prior to the EC-SERS experiment. The collected purified bacterial pellets were each re-suspended in the working T-buffer solution. For in-situ EC-SERS experiments, 80-150 μL of the analyte-containing supporting electrolyte was used to conduct EC-SERS.

### Spectroelectrochemical and SERS Studies

Evaluation of daily variation of the laser power intensity, as well as frequency (wavenumber) calibration of the Raman spectrometer was routinely done as a preceding step, prior to commencing the SERS/EC-SERS experiments, by using known Raman probes, such as polystyrene, cyclohexane and silicon wafer, etc. An aqueous suspension of the bacteria in TrisHCl buffer (prepared as described above) was used in all EC-SERS experiments involving bacterial work. All mycobacterial preincubation experiments were done in TrisHCl buffer (200 mM). All EC-SERS spectra were obtained using medium laser power, which corresponds to 25 mW. Approximately 80-150 uL supporting electrolyte was required to cover the integrated working, auxiliary and pseudo silver reference electrodes. All EC-SERS work was initiated by obtaining a SERS spectrum in the supporting electrolyte (i.e. before connection of the integrated electrodes to the potentiostat), and a SERS spectrum was recorded at open circuit potential, which represented the SERS spectrum in the absence of applied voltage. For the voltage stepping protocol, a specific voltage stepping sequence which involved both anodic and cathodic voltage stepping, as described below, was used. SERS spectra were collected at each step (15 s acquisition). All samples were evaluated at multiple locations across the nanometallic surface. Raw SERS data sets were processed with the freely available algorithm,

Goldindec, for baseline correction. As Goldindec was originally prepared for use in MATLAB, which is a closed-source software, the script was modified for use in Octave (GNU), an open source software. The modified script is available for download from Source forge. Where necessary, for plotting comparison of spectra, all recorded spectra were corrected for laser power intensity and acquisition/integration time.

### Sputum Experiments

The sputum used in this investigation was obtained from the sputum bank at the Lung Institute in Groote Schuur hospital, Cape Town, South Africa. For the relevant sputum-spiking and/or control experiments, sputum samples were from clinically diagnosed TB-negative/HIV-negative, or TB-negative/HIV-positive individuals. Sputum samples from cohorts of clinically diagnosed TB positive samples were used for TB-detection in clinical samples. The sputum was stored at −20° C. and allowed to thaw to ambient temperature immediately prior to use. Prior to the sputum spiking experiments, multiple sputum samples were pooled together, mixed and aliquoted into 500 μL sample aliquots. To prepare for the sputum spiking with the target mycobacteria, an aliquot of a ≈0.8 OD₆₀₀ culture suspension of wtBCG was pelleted by centrifugation. An aliquot of the pelleted concentrate was spiked into aliquot of sputum, followed by very gentle vortex mixing.

### Antibodies

Before immobilization of the antibody, the top-layer of the nanometallic film is further modified with an optically transparent layer of PSS polyelectrolyte, the reaction of which is done in the presence of dilute salt solution, followed by rinsing and drying. This is then followed by interaction of the antibody in HEPES buffer at pH 7.4, which enables the Ab to interact with the nanometallic feature.

### Results and Discussion

### Characterization of the Silver Nanoparticles and Nano-peAgSPc Substrates

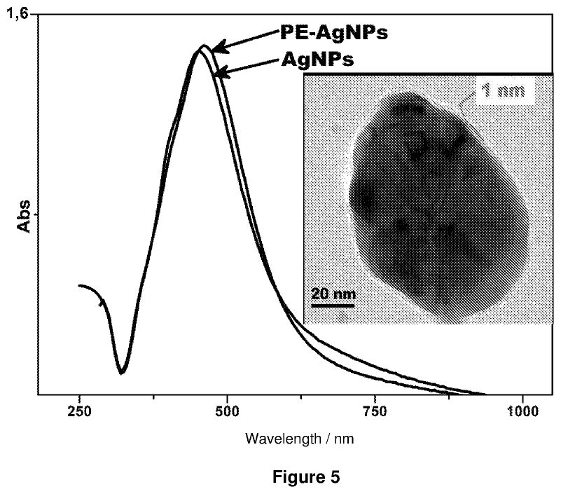

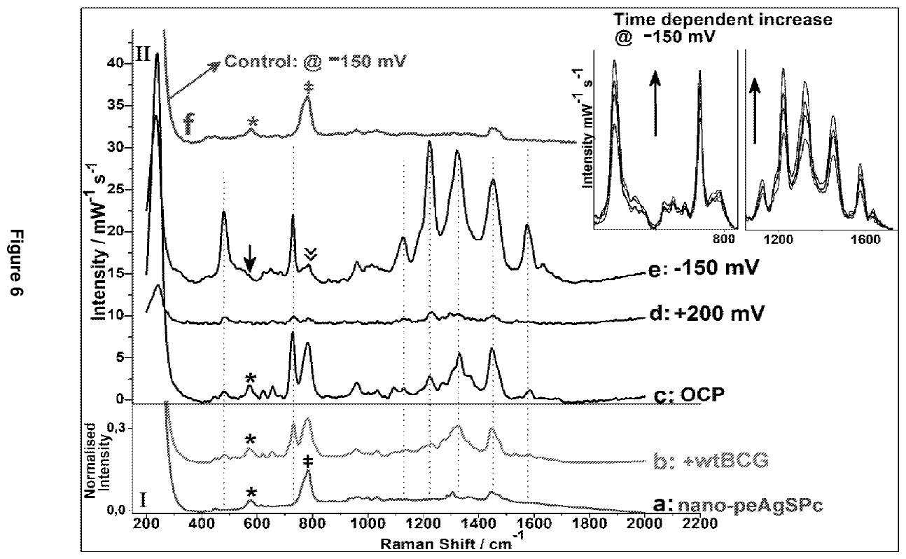

The polyelectrolyte (PE) wrapping of the silver nanoparticles (AgNPs) was not preceded by any SAM passivant layer. The net negative charge of the aqueous colloidal suspensions, as incurred through the Tollen's synthesis process enabled direct PDADMAC wrapping around the surface of the AgNPs. FIG. 5 exhibits the plasmon absorption band for the as-synthesized silver nanoparticle suspensions (see indicated curve), and the pe-wrapped silver nanoparticle suspensions (see indicated curve), i.e. AgNP; peAgNP, respectively. Each plasmon absorption band is characterized by a single peak maximum, which in conjunction with the maxima positions are indicative of quazi-spherical silver nanoparticles. Moreover, unlike for the un-modified AgNP, a small, but distinct increase in the band intensity, as well as red shifting of the position of the plasmon absorption band representing the peAgNP, is clearly visible (i.e. from 450 to 460 nm). The shift to longer wavelength may be ascribed to the change in the localized refractive index of the medium surrounding the surface of the nanoparticles, caused by the adsorption of the charged polyelectrolyte. Furthermore, based on previous research, the small increase in the intensity of the plasmon band absorbance indicates that the layer surrounding the metal core increased in thickness, which is probably due to the presence of the polyDADMAC layer. Additionally, direct visualization of the polyelectrolyte coating on the AgNP surface was achieved through Transmission Electron Microscopy (TEM) analysis. The inset in FIG. 5 illustrates the TEM micrograph of the PE-wrapped AgNP (peAgNP), taken at high magnification, and the PE layer, visually distinguishable as a very faint layer surrounding the darker AgNP can be seen. The +/−˜10 nm peak shift observed from the spectrophotometric analysis, in conjunction with the estimate from high magnification TEM analysis, corresponds to an approximate shell thickness layer of refractive index 1 and thus a thickness of approximately 10 Å for the as-modified peAgNP. The multicycle centrifugal post-processing/washing treatment enabled sufficient thinning of the PE-coating to ensure laser/optical transparency for use in EC-SERS experiments.^{5,6} The positively charged peAgNPs assembled into highly dense/compact and homogenous films, as seen from scanning electron microscopy (SEM) imaging (not shown). These substrates, which include the pre-formed peAgNP film on disposable carbon screen printed electrode (cSPE), are referred to herein as nano-peAgSPc.

### Method Development

To validate the EC-SERS method described herein, experiments were initially performed using intact, whole-cell wild type Mycobacterium bovis BCG (i.e. an attenuated strain of BCG), (wtBCG), because of its structural similarity to the MTB complex. To optimize the sequence voltage stepping protocol of the EC-SERS technique, the progression of signal evolution in association with stepping of the applied voltage (E) was systematically monitored via simultaneously recorded SERS-spectra, which included the appearance, and intensity variation, of bacteria-specific vibrational mode bands, as well as shifting of band positions. The focus during the method development included both pre-exposure/preincubation with the target mycobacteria, followed by EC-SERS (done in absence of any bacteria); as well as in-situ EC-SERS, in which case the EC-SERS was done in the presence of the target bacteria without any preincubation. The former approach is referred to herein as the ecSERS-approach-1 (denoted as incbECSERS-1) (FIG. 3), whereas the latter approach is referred to herein as the EC-SERS-approach-2 (denoted as instECSERS-2) (FIG. 4). All EC-SERS/SERS results shown herein have only been corrected for the spectral response of the system and baseline effects attributed to the 785 nm radiation scattered from the nanostructured substrate itself, and no additional smoothing was applied to any spectra. Potential interference from culture/growth media-related constituents were precluded by a triplicate centrifugal pre-wash process, which was used for all bacteria species/strains.

With reference to the incbECSERS-1 method, FIG. 6 illustrates the development of the SERS spectral features before and after exposure of the nano-peAgSPc platform to the wtBCG mycobacteria, as well as in response to the influence of the voltage stepping during EC-SERS. The SERS spectra were thus chronologically recorded in the exact order in which the experiment was conducted. The spectra in the main graph were offset vertically for ease of viewing. Curve a exhibits the spectrum recorded in air, prior to any exposure of the nano-peAgSPc to the mycobacteria, and here a band at ca. ≈784 cm⁻¹, denoted as “‡”, which, based on literature, may be assumed as stemming from the ring vibration due to the presence of the PDADMAC on the surface of the AgNPs, is seen as the most prominent feature. Notably, the ring structure is all along the polymer chain, and it contains the quaternary ammonium group, which confers the positive charge to the PE, and is thus anticipated to be involved in the initial interaction with negatively charged functional groups on the surface of the bacterial cell wall. Additionally, two other lower intensity bands, all of which are affiliated with other weak SERS modes directly stemming from the PDADMAC-wrapped AgNP (peAgNP) background, is also observable, as seen in Curve a. Curve b shows the spectrum recorded after incubation of the nano-peAgSPc with the wtBCG mycobacteria, which looks distinctly different, as compared to the spectral features of Curve a. In this case, the development of a new band, proximal to the BG-related “‡” band, is clearly distinguishable. The appearance of the band at the ca. ≈50 cm⁻¹ downshifted (redshifted) frequency position, is accompanied by an associated reduction in the peak intensity of the “‡” denoted band. Two additional bands distal to the new band are also clearly visible. Based on the observed peak positions of these new bands and taking into account the SERS peak positions found in different published reports for bacterial related SERS-based literature, the evolution of these bands was assumed to be related to the interaction of specific biochemical structures on the target mycobacterial cell wall with the nanometallic surface, and thus marker bands, indicating the presence of the wtBCG mycobacteria proximal to the surface of the nano-peAgSPc.

FIG. 6c-e exhibit the spectra recorded in mycobacteria-free supporting electrolyte during EC-SERS. More specifically, Curve c shows the SERS spectrum recorded at open circuit potential (OCP), whereas Curves d and e illustrate the SERS spectrum recorded during each applied potential, for the sequence-based positive-to-negative voltage stepping, i.e. at +200 mV (Curve d) and at −150 mV (Curve e), respectively. During OCP, taken in the presence of the neat supporting electrolyte (Tris-HCl, pH 8.5), the electrodes were connected to the potentiostat but no voltage was applied yet, and as seen in Curve c, there is a distinct increase in peak intensity for all bacteria-related marker bands. While the development of additional bands can also be observed, the peak intensities of these additional bands are all still very low at this point. Each of the SERS spectra recorded during the sequence-based voltage stepping, i.e. FIGS. 6d and e, exhibits a distinctly different spectrum. Under the influence of the anodic voltage, i.e. +200 mV (the first applied voltage within the sequence), there is an instant, dramatic reduction in all band intensity maxima and the spectrum becomes almost featureless. Under the influence of the cathodic voltage on the other hand, all bacteria-related bands reappear, with almost all bands now exhibiting a marked increase in peak intensity (by orders of magnitude), as compared to all previous spectra. Notably, the mode band originally denoted “‡”, has now been replaced by a markedly upshifted (blueshifted) band, in association with a significantly reduced peak intensity (see indicated band @−150 mV, Curve c). Interestingly, the reduction/frequency shift of the “‡” denoted BG-related band, in association with interaction with target bacteria and/or tuning of the applied voltage does not happen in the absence of the mycobacteria (i.e. in control studies). In view of the PE itself, reduction of the band maxima and/or frequency shift representing this mode (ring structure along the polymer chain) would signify opening of the ring. However, given that this phenomenon (band reduction/frequency shift) does not happen in the absence of the mycobacteria (control studies), clearly the presence of the mycobacteria has a marked effect on the immediate vicinity of the nanometallic surface. Interaction with charged groups on the surface of the bacteria cell wall could have affected electrophilic attack of the charged group in the ring structures, thus inducing ring opening, which may be intensified in conjunction with the voltage stepping during EC-SERS. Alternatively, it could be that the vibrational mode from the PE is masked by the close proximity of the mycobacteria to the nanometallic surface through specific, irreversible adsorption, driven by the follow-up EC-SERS. Still with reference to the cathodic voltage, the band at ca. ≈479 cm⁻¹ (visible here as one of the most prominent features within the spectral pattern for wtBC), is of particular significance, since it was shown to be one of the most distinctive vibrational modes associated with the signal for the TB-affiliated/TB-derived mycobacteria, and thus not observed for other bacterial types.

Control experiments were also carried out on the supporting electrolyte itself, as well as to assess the possible contributions of the cell growth media. Notably, in the case of the control (i.e. in the absence of any bacteria on the nanometallic surface), as shown in Curve f of FIG. 6, under the influence of the cathodic voltage (i.e. −150 mV), the spectral signature is the unequivocally similar as observed for the neat peAgSPc. This was seen both in the case in which the control was preincubated with the cell culture media, followed by EC-SERS; as well as when the control was directly used for EC-SERS without preincubation, both of which indicate that neither the growth medium nor the electrolyte itself contributed to the observed spectral features obtained for the wtBCG.

The potential dependent SERS spectral variation showed that the chemical (i.e. charge transfer, CT) enhancement mechanism was cooperative with the electromagnetic enhancement (EM) mechanism in the EC-SERS system. Moreover, the long-range EM-and short-range, chemical enhancement (CE) mechanisms were not mutually exclusive, but operated in concert to provide the overall SERS effect. On the other hand, the CE enhancement mechanism reflects enhancements resulting from chemical interaction between the metallic surface and the target adsorbate, with the type of CE enhancements ranging between (i) chemical bonding, (ii) resonance enhancement of a surface complex, and (iii) photon-induced substrate-to-adsorbate/adsorbate-to-substrate charge transfer (PI-CT).

The positive voltage was insufficient to produce the photon-driven CT states on the metal surface with an excitation energy of hv, and the change in the relative SERS signal at this E may primarily be due to intensifying of the metal-adsorbate interaction, i.e complex formation/bonding effect. Consideration also needs to be given to the possibility of the refractive index change around the nanometallic surface, as incurred by possible surface metal complex formation/surface-bound bacteria-adsorbate. In contrast, for the negative voltage, apparent characteristic vibrational (SERS) modes immediately following the cathodic stepping of the applied potential may be a manifestation of the charge density dependent plasmon frequency shift, usually effected by increase in and blue-shift of the plasmon resonance band. Additional time-dependent studies revealed a rapid increase in all major bacterial representative band intensity maxima, as the negative voltage was maintained, for up to several minutes. The Insets of FIG. 6 exhibit the intensity increase of all major bands during time-dependent EC-SERS, recorded consecutively, while holding the potential at −150 mV, up to 120 s, and the distinct increase in signal (band) intensity with time is clearly visible. Based on previous findings, the time-dependent, rapid/distinct increase in band intensities at this (negative) voltage may also be an indication of a mild change in the bonding strength of the bacteria-adsorbate, interacting with the charged surface, or may indicate that the process is potentially dominated by the CT enhancement mechanism.

No further increases in any bacteria-related band peak intensities were observed beyond this negative voltage. In fact, a stark decrease in band intensities was observed with further negative movement of the applied voltage (results not shown). While the aforementioned time-dependent signal increase at −150 mV is indicative of CT enhancement, driven by the increase in the Fermi level of the metallic surface, the signal reduction observed beyond −150 mV suggests a deviation from the ideal resonance-like Raman scattering condition beyond that voltage limit.

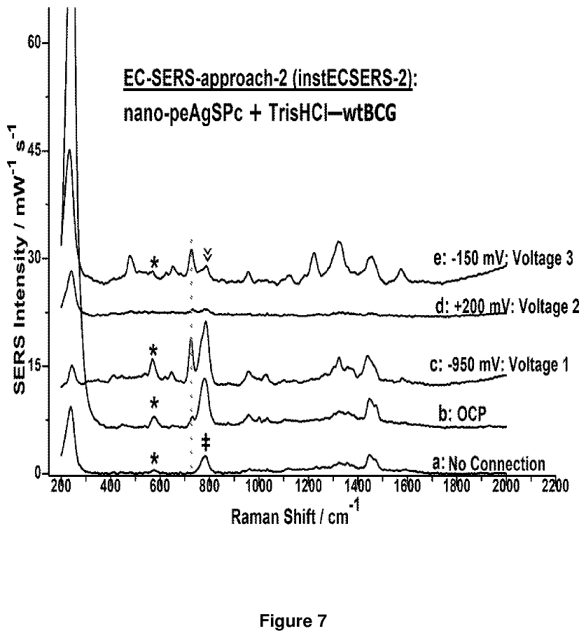

With reference to the instECSERS-2 method, FIG. 7 depicts the results. As stated earlier, the sequence-based positive-to-negative voltage stepping was done in the presence of the mycobacteria contained within the supporting electrolyte, without any preincubation/pre-exposure. In this EC-SERS approach, the voltage stepping was preceded by initial polarizing at a cathodic voltage of −950 mV (referred to as the “conditioning step”). While the conditioning step was not critical for obtaining a viable signal of the target mycobacteria, it's inclusion within the EC-SERS protocol for the instECSERS-2 approach effected improved reproducibility across the nanometallic surface, and better repeatability between platforms. The SERS spectra were chronologically recorded in the order in which the experiment was conducted. Curve a of FIG. 7 show the SERS spectrum obtained immediately after the peAgSPc was submerged in the Tris-HCl-wtBCG buffer suspension, i.e. before connecting the integrated electrodes (working-, pseudo reference- and counter electrodes) to the potentiostat. Curve b exhibits the SERS spectrum recorded at OCP. FIGS. 7c and d illustrate the SERS spectrum recorded during each applied potential for the sequence-based positive-to-negative voltage stepping (i.e. at +200 mV and at −150 mV, respectively). Similarly to what was observed in incbECSERS-1, the development of a new band, proximal to the BG-related “‡” band occurs early on, faintly visible in this case during OCP. However, under the influence of the cathodic voltage, i.e. during the conditioning step, the peak intensity of this mode increases, here also with an associated decrease in the “‡” denoted BG-related band peak. Additional bands of other vibrational modes, distal to the new band, are also visually distinguishable at this potential. Under the influence of the sequence-based voltage stepping, a similar trend as compared to incbECSERS-1 was observed. Consequently, at +200 mV, the observed SERS spectral response became featureless, while the onset of the negative potential was accompanied by an immediate reappearance of bacterial-related bands, including appearance of additional vibrational mode bands, all with an associated intensity increase. Under the influence of the negative voltage, the mode band originally denoted “‡”, has once again been replaced by a markedly upshifted (blue-shifted) band, in association with a significantly reduced peak intensity (as indicated/denoted in the Curve @−150 mV). It is important to note that aside from some marginal variation in exact band maximum frequency/intensity, as compared to spectra observed during the incbECSERS-1 method, the core integrity of the spectral signature was maintained in both EC-SERS approaches.

### Signal Reproducibility and Surface Characterization

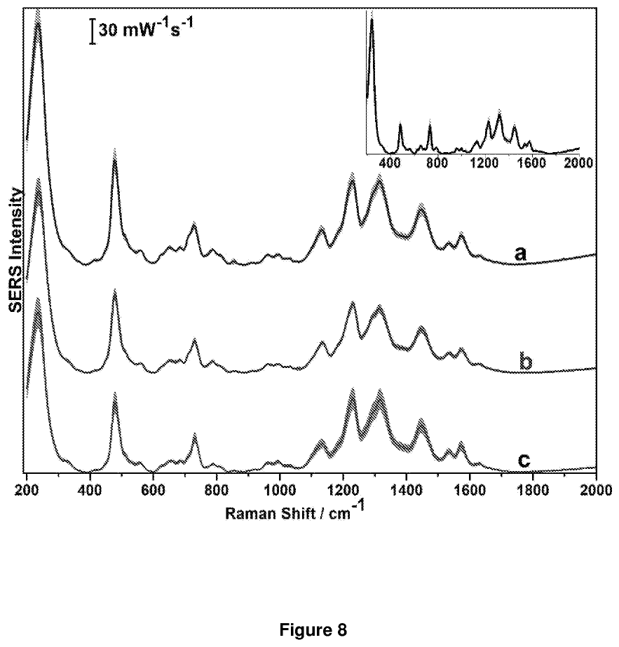

Focusing on the incbECSERS-1 method (EC-SERS-approach-1), and under optimized conditions, the reproducibility of the EC-SERS technique was evaluated through a multifactorial approach, including substrate reproducibility, variation in culture conditions, and spot-to-spot spectral pattern comparison. For the reproducibility studies, each exhibited SERS spectrum represents the mean spectrum, averaged from multiple spectra (at least 12-15). FIGS. 8a-c show the SERS signal for wtBCG, obtained for 3 separate trials (with 3 separate platforms), and the reproducibility between the 3 spectral patterns is self-evident. The inset in FIG. 8 illustrates the SERS spectrum for wtBCG obtained through insECSERS-2, shown here for comparison purpose. The characteristic spectral pattern compares well with that observed in the case of that obtained using the incbECSERS-1 approach.

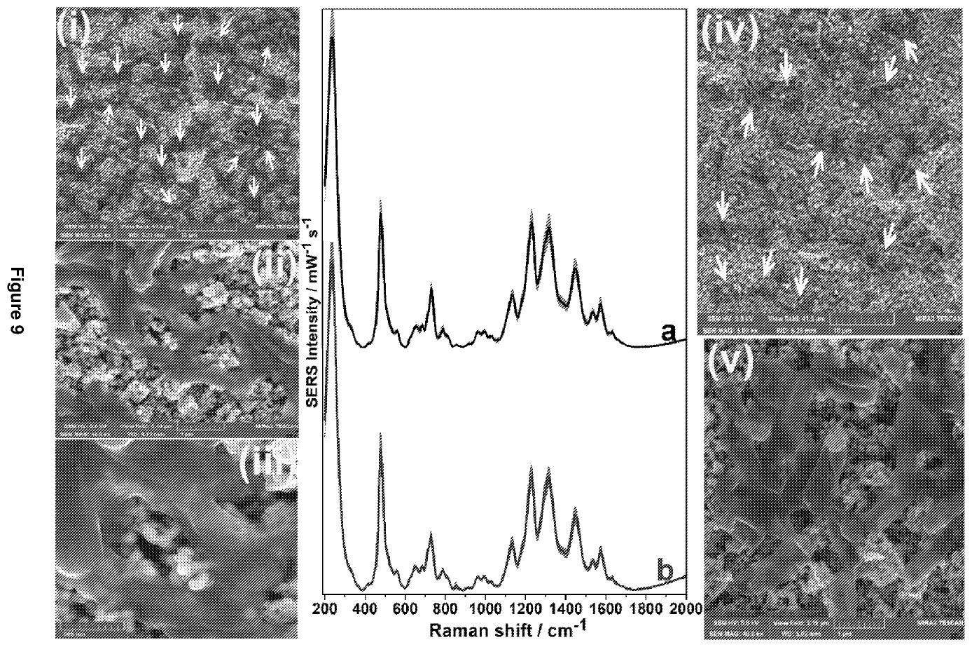

The effects of variation of cell culture conditions on the signal for wtBCG are shown in FIG. 9. Curve a shows the SERS spectrum obtained for the mycobacteria derived from laboratory cultures grown in the absence of polysorbate-80 (tween-80, referred to herein as 7H9-media-A). Curve b exhibits the signal obtained for wtBCG derived from lab cultures grown in the presence of tween-80 (referred to herein as 7H9-media-B). The reproducibility between the two SERS spectra is clear and evident. The same representative vibrational modes are observed in both spectral patterns, which accentuates the reproducibility of the EC-SERS-based detection method.

Morphological/topographical characterization of the mycobacteria on the silver nanometallic platform for the culture-variation experiments was also performed. Images i-v of FIG. 9 show the scanning electron microscopy (SEM) images of the mycobacteria on the nano-peAgSPc platform. Images i-iii illustrates the SEM images obtained for wtBCG grown/isolated from 7H9-media-B; whereas in the case of Images iv-v, the wtBCG mycobacteria was derived from cell culture media that excluded polysorbate. At low magnification (i.e. ≈5 kx), the general distribution of the mycobacteria across the nanometallic surface can be observed, as indicated by the arrows shown in Image i and Image iv. At higher magnification (˜40 kx and 100 kx), i.e. Images ii-iii and Image v, a more distinctive view of the rod-shaped bacilli are observed. The mycobacteria cells originally obtained from 7H9-media-B appear more elongated, whereas the mycobacteria isolated from 7H9-media-A appear flatter. These SEM images also confirm the presence of the bacteria on the nano-peAgSPc platform, since control samples subjected to the same sequence-based electrochemical voltage stepping protocol (i.e. excluding bacteria) exhibited SEM images completely devoid of any bacteria-resembling/related surface constituents. Moreover, while the high waxy content in the cell wall of mycobacteria is known to typically induce coiling between bacilli, thus forming interconnected features such as seen here at higher magnification, the intimate contacts between neighboring bacterial cells and the underlying nanometallic surface also resembles cohesion, a type of ‘cementing’, which is ubiquitous in strong surface bacterial adhesion. Furthermore, as particularly observed in Images iii and v, the intimate contacts between the immobilized bacilli and the nanometallic surface were such that the bacilli appeared stretched, almost to the point of a degree of transparency, which enabled the outline of the underlying silver nanoclusters to be visually distinguishable on these images. Collectively, these images point to the strong binding force between the adsorbed mycobacteria and the nano-peAgSPc platform. The nanometallic silver film had a large surface area, providing excess numbers of available binding sites; the total binding force between bacterial surface appendages and high binding-site surfaces has been described as exceeding that of covalent bonds; and the static EC-field generated via EC-SERS is powerful, creating a large field-gradient. Consequently, capped nanoparticles may be able to partition through outer layers of the cell envelope to reach/bind the plasma membrane.

To evaluate signal uniformity of the bacterial cells across the nanometallic surface, SERS spectra were recorded at various locations across the surface (not shown). From the spot-to-spot spectral comparison, all prominent vibrational mode bands are reflected across the entire nanometallic surface, thus exhibiting an overall reproducible spectral pattern across all spots. This illustrates the sensitivity and reproducibility of the method described herein in species-specific identification of target mycobacteria.

### Tentative Band Assignment

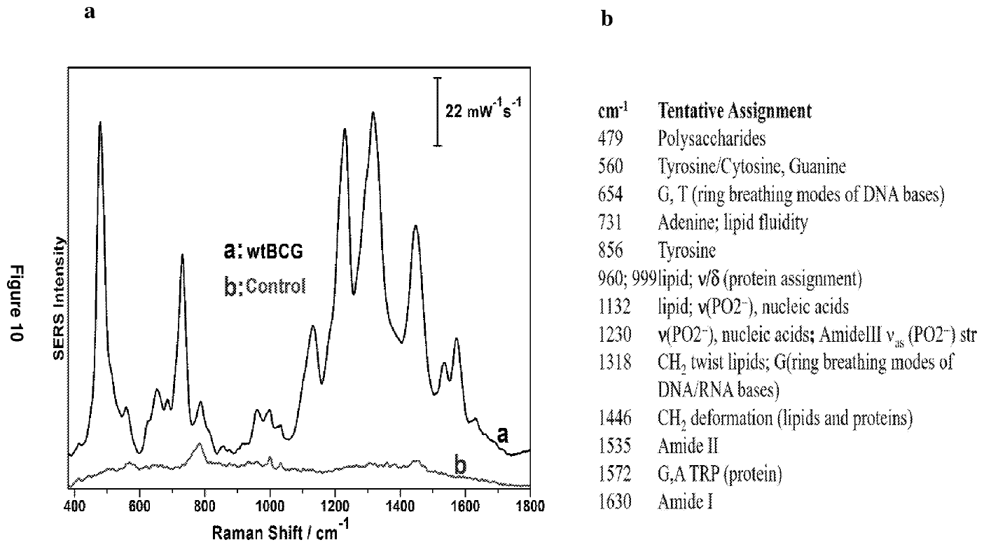

FIG. 10a (Curve a) shows a detailed expansion (i.e. shown at a higher scale resolution) of the characteristic vibrational mode bands/spectral pattern obtained for the wtBCG mycobacteria, from the EC-SERS technique. The characteristic SERS spectrum for the control (Curve b) is also shown for comparative purpose. No Raman spectra for the bacteria is shown, since the vibrational modes were too weak to obtain any distinguishable spectral pattern during spontaneous (bulk) Raman analysis of the native bacteria. Since no distinguishable bulk Raman spectra of the mycobacteria could be obtained, a quantitative measure of the magnitude of the SERS enhancement for wtBCG could not be determined. However, since all the prominent SERS bands for the characteristic SERS spectrum of wtBCG could be assigned according to published data,⁷ a tentative band assignment for the represented vibrational modes was tabulated and is shown in FIG. 10(b) and Table 1.

Prior studies involving silver-based EC-SERS/SERS research suggest that positive voltages be avoided due to the marked negative impact on and/or irreversible loss of SERS activity of the nanostructured platform. However, this was not the case in the current study. On the contrary, the anodic stepping of the applied voltage was important for the EC-SERS technique in this study. Moreover, additional experiments involving incremental increase of the positive voltage (within the context of the sequence-based positive-to-negative voltage stepping protocol), in 100 mV increments (for up to +500 mV), exhibited a marked increase in all characteristic band maximum peak intensities, in each case, observed from the SERS spectra recorded during the cathodic stepping of the voltage (i.e. −150 mV). This phenomenon was observed for both EC-SERS approaches (results not shown), and again, on condition of not being bound by any specific mechanism of action and/or theory, this unexpected divergence from what has previously been described may be due to the following: Firstly, oxidation has been noted as one of the key chemical reactions to effect irreversible adsorption during the natural bacterial cell-substratum adhesion process. Thus, considering the low energy of activation for desorption of bacteria during initial stages of adsorption, the influence of the positive/anodic voltage within the generated static EC-field may have alleviated the energy barrier that would otherwise have incurred between the bacterial cell wall and the nanometallic surface within the electrochemical double layer. Furthermore, the numerous physicochemical properties of charged polyelectrolytes (in this case polyDADMAC), one of which is its innate muco-adhesive property and its associated contribution to platform affinity for the target bacteria, coupled with the EC-effected intensifying of the specific adsorption of the bacteria probably contributed to stabilizing the otherwise labile SERS-active sites while under the influence of the applied voltage during EC-SERS. The presence of quaternary charged amines (found in the ring structure all along the polymer) provides an innate ability to the polymer to resist degradation under oxidative attack—a quality that may have assisted in stabilising the underlying plasmonic nanostructures while under the influence of the oxidative voltage. Moreover, in aqueous samples used in EC-SERS, the water acts as heat sink, while the irradiated volume in EC-SERS is much larger than that of a dry spot on a solid substrate. The reduced heat generation, coupled with the attenuated laser power at the source, possibly contributed to minimizing thermal- and/or photodegradation of the thin optically transparent stabilizing PE-layer during the EC-SERS experiments, thus minimizing destabilization of the silver nanoclusters and associated labile SERS sites.

The above results show that the EC-SERS technique described herein is suitable for detection and identification of a mycobacterium target probe on the nanometallic surface. As wtBCG is a surrogate representative for ‘pathogenic’ mycobacterium TB, it follows that the same method could also be used to detect MTB.

Detection of Mycobacterium tuberculosis (MTB)

The strains of mycobacterium tuberculosis (MTB) used in this study include TB-H37Rv, TB-HN878, and TB-CDC1551, which are clinically relevant hypervirulant strains of MTB. The results described herein are all based on the incbECSERS-1 method, which as previously described involves pre-exposure to the target bacteria, followed by EC-SERS. Compared to the SERS spectral feature development observed for wtBCG, an unequivocally similar spectral reponse trend was observed for the TB mycobacteria strains. Curves b-d of FIG. 11 show the evolution of the SERS vibrational mode bands, i) after exposure of the nano-peAgSPc to the H37Rv strain of MTB (Curve b), and ii) in association with the follow-up EC-SERS (Curves c-d). The SERS spectrum for the nano-peAgSPc before exposure to the TB-mycobacteria (Curve a) is also shown for comparison purposes. As can be seen from the displayed results, the vibrational modes distinguishable after incubation with the TB mycobacteria and the development of the mode bands in association with the EC-SERS compare well with what was observed in FIG. 6. Notably, as was observed for wtBCG, the vibrational mode proximal to the “‡” BG-related band developed early on during incubation (within the first 10 min), while its band maxima also exhibited a marked increase, coupled with an associated decrease/frequency shift of the “‡” denoted band, under the influence of voltage stepping during EC-SERS. This phenomenon was seen for all MTB strains detected with the EC-SERS technique. This finding corroborates the indication that the charged quaternary amine group found in the ring structure along the polymer chain may be involved in the interaction with negatively charged biochemical structures on the cell wall of the mycobacteria during the initial stages of adsorption. FIGS. 11 II a and b display the SEM images obtained for TB-H37Rv mycobacteria on the nano-peAgSPc, at lower (˜5 kx) and at higher (˜25 kx) magnification, respectively. While the distribution of the mycobacteria across the nanometallic surface can be seen in Graph a, a more distinctive view of the mycobacteria can be seen under the higher magnification.

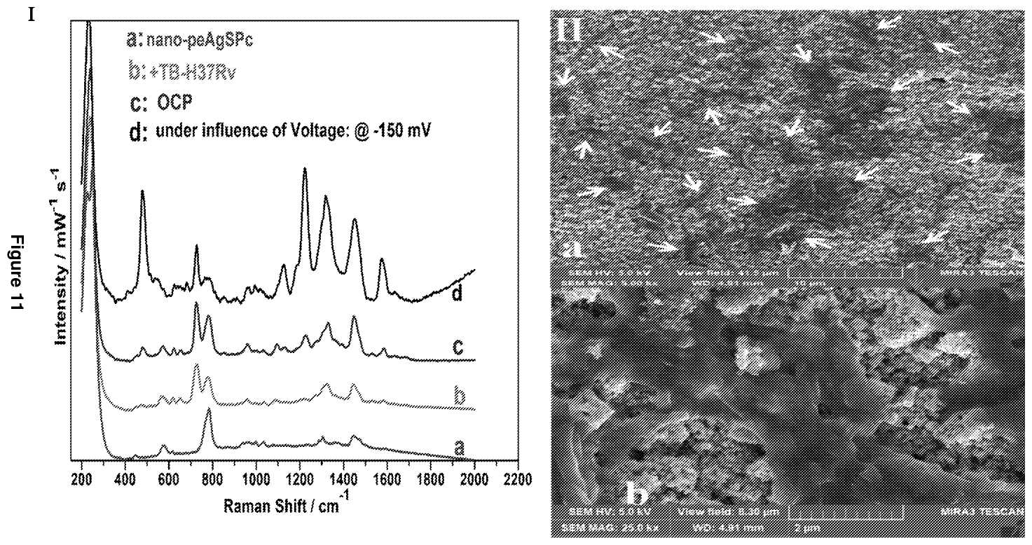

Reproducibility studies for TB-H37Rv were also conducted. FIG. 12a illustrates the SERS signature for TB-H37Rv, obtained for three separate trials using three separate platforms; whereas FIG. 12b depicts the spot-to-spot spectral pattern comparison, recorded from various locations across the nanometallic surface. Apart from subtle variations, on both counts the SERS spectral reproducibility is evident.

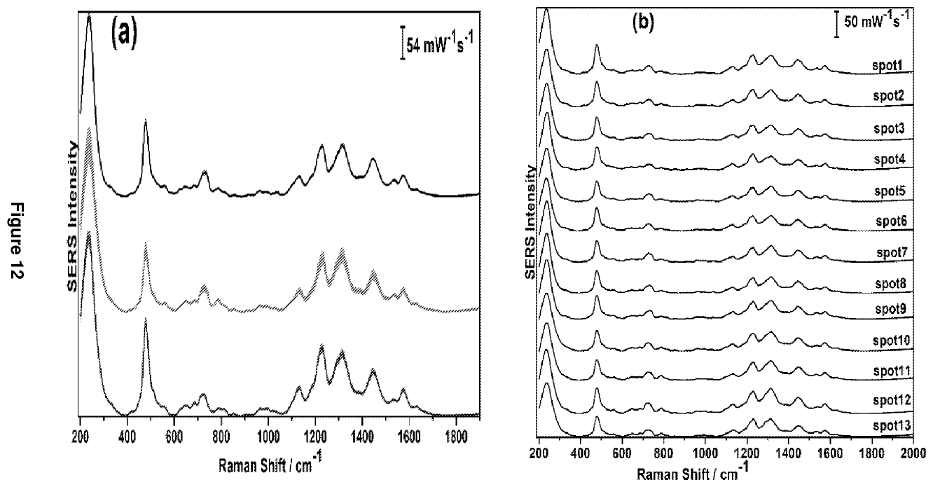

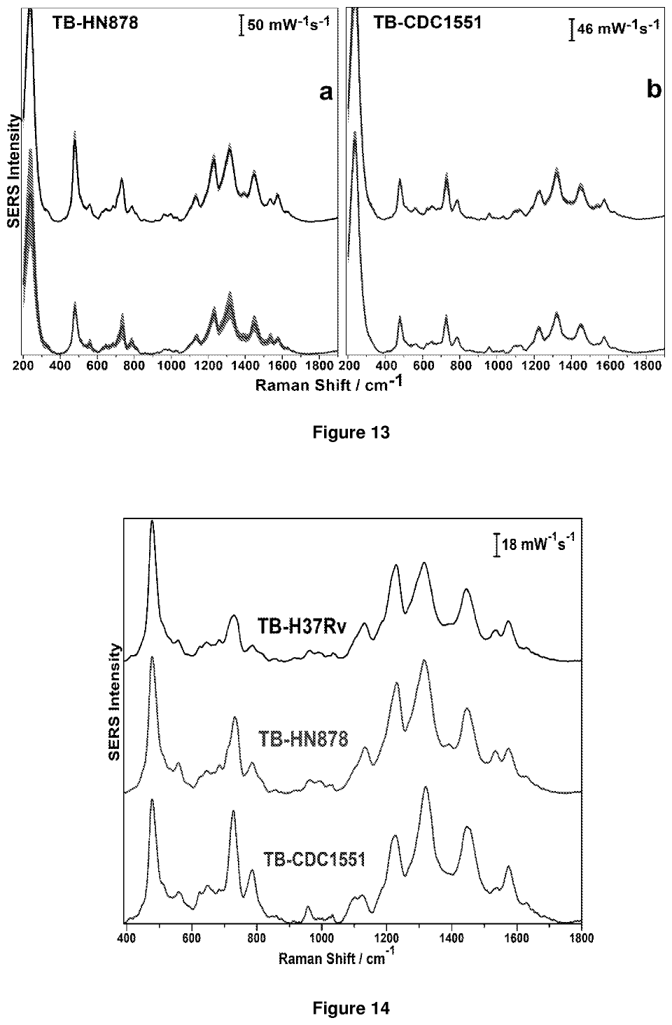

Reproducibility studies were also done for the other two MTB strains investigated in this study, and FIG. 13 exhibits the reproducibility of the SES vibrational signature obtained for TB-HN878 and for TB-CDC1551, for two separate trials using the EC-SERS technique.

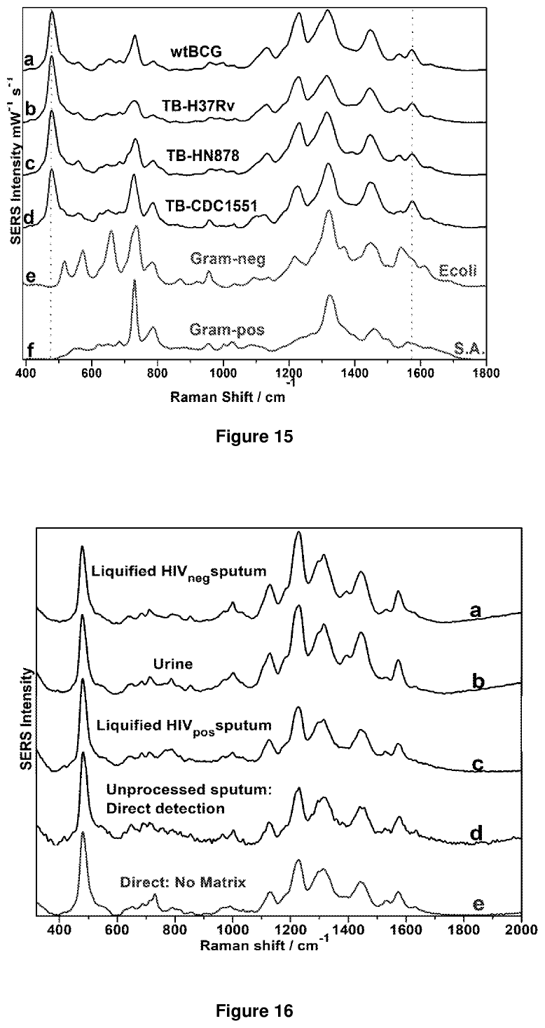

The SERS spectra of all three strains of MTB, obtained through the EC-SERS technique described herein, are shown in FIG. 14. Overall these spectra illustrate the sensitivity of the technique towards detecting TB mycobacteria, while the vibrational signature for each strain is sufficiently unique to provide a fingerprint for strain-level discrimination/identification. Notably, the band <500 cm⁻¹ is commonly seen as a represented vibrational mode in each of the three SERS spectra, and illustrated to be one of the most prominent features in the SERS spectral signature for each strain of MTB studied here. This vibrational mode appears to be unqiue to the TB-affiliated mycobacteria, as it was also observed within the vibrational signature obtained for wtBCG but was not observed for gram-negative or gram-positive bacteria. A graphical illutration of this is seen in FIG. 15, which depicts the SERS spectral signatures for all of the mycobacterial targets used in this study, along with comparitive SERS spectra for gram-negative bacteria (E. coli, K-12 strain), and gram-positive bacteria (Stephylococcus aureus, SA). These results also illustrate the ability of the technique for species-based discrimination. This vibrational mode is thought to be affiliated with the glycoconjugate part of the bacterial cell wall, and more specifically may be attributed to the most abundant extracellular lipopolysaccharide in the mycobacteria, and thus possibly a representative of lipopolysaccharide lipoarabinomannan (LAM).

TABLE 1
Tentative assignments of spectral peaks for bacteria of FIG. 15
Assignments for all Mycobacteria

| | TB- | TB- | TB- | |
|---|---|---|---|---|
| wtBCG | H37Rv | CDC1551 | HN878 | |
| (cm⁻¹) | (cm⁻¹) | (cm⁻¹) | (cm⁻¹) | Tentative assignments |
| 479 | 478 | 478 | 478 | Polysaccharides |
| 560 | 558 | 559 | 559 | Tyrosine/Cytosine, Guanine |
| 653; | 647; | 649; | 647; | Guanine, Tyrosine (ring breathing of DNA bases), Adenine (ring breathing mode of DNA/RNA |
| 658 | 685 | 685 | 686 | bases); Guanine ring breathing, (NAG) |
| 730 | 730 | 728 | 732 | Adenine (ring breathing mode of DNA/RNA bases) |
| 854 | 852 | 860 | 857 | Tyrosine (buried) |
| 962 | 963 | 962 | 957 | Lipid, ν/δ (protein assignment) |
| 1032 | 1035 | 1033 | 1032 | δ(C—H), Phenylalanine (proteins assignment) |
| 1133 | 1132 | 1103; | 1133 | Phenylalanine C—H in plane bending, C—N and C—C stretch, n(PO₂⁻), |
| | | 1126 | | nucleic acids |
| 1230 | 1229 | 1227 | 1231 | ν(PO₂⁻), nucleic acids, Amide III νₐₛ |
| 1315 | 1315 | 1320 | 1315 | CH₂ twist lipids, Guanine (ring breathing modes of DNA/RNA bases), cytosine |
| | | | 1390 | 200200 |
| 1446 | 1445 | 1447 | 1447 | CH₂ deformation (lipids and proteins), CH₂CH₃ deformation, scissoring (fatty acids, |
| | | | | phospholipids, and mono- and oligo-saccharides) |
| 1535 | 1536 | 1539 | 1534 | Amide II (of proteins), Amide II of N-acetyl related bands |
| 1572 | 1574 | 1575 | 1574 | Guanine (G), Adenine (A) TRP (protein) ring stretching |
| 1630 | 1630 | 1630 | 1630 | Amide I, carbonyl stretching |

Assignments for gram negative (Gr⁻) and gram positive (Gr⁺) bacteria

| EColi (Gr⁻) (cm⁻¹) | S.A. (Gr⁺) (cm⁻¹) | |
|---|---|---|
| 517 | | ν(S—S) disulfide (amino acid cysteine) |
| 573 | | Tyrosine/Cytosine, Guanine |
| | 622 | C—C twisting mode of phenylalanine (skeletal) |
| 659 | 649 | Guanine, thymine ring breathing, A (ring breathing mode of DNA/RNA bases) |
| 735 | 730 | Adenine (ring breathing mode of DNA/RNA bases) |
| 869 | | Polysaccharides |
| 956 | 956 | νₛ(CH₃) of proteins, C—C stretching |
| 1035 | 1028 | δ(C—H), Phe (proteins assignment), C—H in plane, C—N Gly |
| 1093 | 1086 | νₛ(PO₂⁻) in nucleic acid |
| 1139 | | ν(PO₂⁻) in nucleic acid, (C—C) skeletal str in alkane |
| 1218 | | Stretching vibration (C—N) |
| 1322 | 1324 | G (ring breathing modes of DNA/RNA bases), CH₂ twist (lipids) |

Detection of Mycobacteria from Human Sputum

To illustrate the translational potential of the method described herein toward point of care (POC) settings, the clinical adaptibility with reference to sputum-and urine based biological milieu were investigated. Sputum samples from already banked cohorts, already characterized and clinically diagnosed as TB-negative, were spiked with wtBCG as a surrogate TB-representitive. The clinically relevant biological samples included HIV_{neg} sputa, HIVₚₒₛ sputa, and HIV_{neg} urine. For these experiments, the nanostructured platform was further derivatized with an antibody (Ab) that specfically targets mycobacterium-based antigens as biological capture agent. In the case of the liquefied sputum, following the release and isolation of the mycobacteria from the liquefied sputum, the Ab-derivitised nanometallic platform (Ab-nano-pe₂AgSPc) was then exposed to the mycobacteria for capturing during the pre-incubation step. In the case of spiked sputum that was not subjected to liquefaction, Ab-nano-pe₂AgSPc was exposed to the wtBCG-spiked sputum for capturing during the pre-incubation step. In all cases, the capture step was subsequently followed by follow-up EC-SERS. As previously described, FIG. 16a-d exhibits the SERS signal obtained for these studies (i.e. with sputum and urine). The signal obtained in these studies compared very well with that obtained witout any sputum or urine matrix (Plot e of FIG. 16).

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

### References

(1) Lynk, T. P.; Sit, C. S.; Brosseau, C. L. Electrochemical Surface-Enhanced Raman Spectroscopy as a Platform for Bacterial Detection and Identification. Anal. Chem. 2018, 90, 12639-12646.
(2) Novotny, R. The Influence of Complexing Agent Concentration on Particle Size in the Process of SERS Active Silver Colloid Synthesis. 2005, 1099-1105.
(3) Sharma, V. K.; Yngard, R. A.; Lin, Y. Silver Nanoparticles: Green Synthesis and Their Antimicrobial Activities. Adv. Colloid Interface Sci. 2009, 145, 83-96.
(4) Panacek, A.; Libor, K.; Prucek, R.; Kolar, M.; Vecerova, R.; Pizurova, N.; Sharma, V. K.; Nevecna, T.; Zboril, R. Silver Colloid Nanoparticles: Synthesis, Characterization, and Their Antibacterial Activity (Tollens Reagent Synthesis Method). J. Phys. Chem. 2006, 110, 16248-16253.
(5) Chauvet, R.; Lagarde, F.; Charrier, T.; Assaf, A.; Daniel, P.; Chauvet, R.; Lagarde, F.; Charrier, T.; Assaf, A. Microbiological Identification by Surface-Enhanced Raman Spectroscopy. Appl. Spectrosc. Rev. 2017, 52, 123-144.
(6) Israelsen, N. D.; Hanson, C.; Vargis, E. Nanoparticle Properties and Synthesis Effects on Surface-Enhanced Raman Scattering Enhancement Factor: An Introduction. Sci. World J. 2015, 1-12.
(7) Lemma, T.; Saliniemi, A.; Hynninen, V.; Hytönen, V. P.; Toppari, J. J. SERS Detection of Cell Surface and Intracellular Components of Microorganisms Using Nano-Aggregated Ag Substrate. Vib. Spectrosc. 2016, 83, 36-45.

1. A method of detecting a microorganism in a sample, the method comprising the steps of:
applying a sample to a Surface Enhanced Raman Scattering (SERS)-active surface comprising an electrode which is coated with a film of polyelectrolyte-wrapped noble metal nanoparticles;
applying a first voltage and then a second voltage in a step-wise manner to the SERS-active surface;
generating a SERS spectrum of the SERS-active surface; and
determining whether the generated SERS spectrum or part thereof is characteristic for the microorganism.

2. The method according to claim 1, wherein the first voltage is an anodic voltage and the second voltage is a cathodic voltage.

3. The method according to claim 1, which further comprises the step of allowing the microorganism to be captured to the SERS-active surface if the microorganism is present in the sample, prior to the step of applying the first and second voltages.

4. The method according to claim 1, wherein the film of polyelectrolyte-coated nanometallic particles is functionalised with capture agents which specifically recognise and capture the microorganism onto the SERS-active surface.

5. (canceled)

6. The method according to claim 1, wherein the noble metal nanoparticles are silver nanoparticles.

7. The method according to claim 1, wherein the polyelectrolyte is poly(diallyldimethylammonium chloride) (PolyDADMAC).

8. The method according to claim 1, wherein:
the first voltage is less than or equal to +600 mV; and/or
the second voltage is less negative than or equal to −300 mV.

9. (canceled)

10. (canceled)

11. (canceled)

12. (canceled)

13. The method according to claim 1, wherein the step of determining whether the generated SERS spectrum or part thereof is characteristic for the microorganism is performed by comparing the generated SERS spectrum to a reference SERS spectrum of the target microorganism.

14. The method according to claim 1, wherein the step of determining whether the generated SERS spectrum or part thereof is characteristic for the microorganism is performed by identifying one or more vibrational mode bands in the generated SERS spectrum which are known to be characteristic for the target microorganism.

15. The method according to claim 1, wherein the microorganism is selected from the group consisting of a bacterium, a virus or a parasite.

16. The method according to claim 15, wherein the microorganism is Mycobacterium tuberculosis.

17. (canceled)

18. (canceled)

19. (canceled)

20. (canceled)

21. A SERS-active surface comprising an electrode which is coated with a film of polyelectrolyte-wrapped noble metal nanoparticles.

22. The SERS-active surface according to claim 21, wherein neither the electrode nor the nanoparticles are coated with a self-assembled monolayer (SAM).

23. The SERS-active surface according to claim 21, wherein the nanoparticles are silver nanoparticles.

24. The SERS-active surface according to claim 21, wherein the polyelectrolyte wrapping the metal nanoparticles is poly(diallyldimethylammonium chloride) (PolyDADMAC).

25. The SERS-active surface according to claim 24, which comprises an electrode coated with a first polyelectrolyte film comprising PolyDADMAC-wrapped nanoparticles and a second polyelectrolyte film comprising polystyrene sulfonate (PSS).

26. The SERS-active surface according to claim 21, wherein the polyelectrolyte-wrapped noble metal nanoparticles are functionalised with capture agents which specifically recognise a target microorganism, the capture agents being selected from the group consisting of antibodies, affibodies, enzymes, ankyrin repeat proteins, armadillo repeat proteins, nucleic acid aptamers, peptides, carbohydrate ligands, synthetic ligands and synthetic polymers.

27. The SERS-active surface according to claim 21, which is a modified working electrode of a screen-printed electrode.

28. (canceled)

29. (canceled)

30. A kit comprising:
instructions for performing the method of claim 1; and
at least one SERS-active surface which is coated with a film of polyelectrolyte-wrapped noble metal nanoparticles;
and optionally:
one or more reference SERS spectra or distinguishing SERS band information of target microorganism(s);
one or more buffer solutions and/or buffer-based supporting electrolytes; and/or
one or more capture agents.

31. (canceled)

32. (canceled)

33. The method according to claim 1, which further comprises the step of administering an effective amount of a medicament for treating an infection of the microorganism to a subject when the microorganism is detected in a sample from said subject.

34. (canceled)
